(19) United States
(12) Patent Application Publication
(10) Pub. No.: US 2026/0107967 A1
(43) Pub. Date: Apr. 23, 2026

(54) AEROSOL GENERATING MATERIAL COMPRISING A SUBSTITUTED 3-(1-METHYLPYRROLIDIN-2-YL)PYRIDINE COMPOUND

(71) Applicant: RAI STRATEGIC HOLDINGS, INC., Winston-Salem, NC (US)

(72) Inventors: Kyle Ford, Pfafftown, NC (US); Brandon S. Darrow, Winston-Salem, NC (US); Michael R. Galloway, Winston-Salem, NC (US); Gary M. Dull, Lewisville, NC (US)

(21) Appl. No.: 19/361,628

(22) Filed: Oct. 17, 2025

Related U.S. Application Data
(60) Provisional application No. 63/708,910, filed on Oct. 18, 2024.
(60) Provisional application No. 63/880,511, filed on Sep. 12, 2025.

(51) Int. Cl. A24B 15/16; A24B 15/32; A24B 15/38; A24B 15/40; A24F 40/20; A61K 31/00; A61K 31/4045; A61K 31/465; A61K 31/522

(52) U.S. Cl. CPC A24B 15/16; A24B 15/32; A24B 15/385; A24B 15/403; A24F 40/20; A61K 31/4045; A61K 31/465; A61K 31/522; A61K 31/658

(57) ABSTRACT

The present disclosure provides an aerosol generating material for use in a non-combustible aerosol provision system. The aerosol generating material includes an aerosol former, optionally a binder, optionally a non-tobacco botanical material, and at least a substituted 3-(1-methylpyrrolidin-2-yl)pyridine, 3-(azetidin-2-yl)pyridine, or 3-(azetidin-2-ylmethoxy)pyridine as an active agent. The aerosol generating material may be in sheet form, shredded form, bead form, fibrous form, or extrudate form. Further provided are aerosol provision systems including the aerosol generating material.

### CROSS-REFERENCE TO RELATED APPLICATIONS

This application claims priority to U.S. Provisional Application No. 63/708,910, filed on Oct. 18, 2024, and claims priority to U.S. Provisional Application No. 63/880,511, filed on Sep. 12, 2025, each of which is incorporated herein by reference in its entirety and for all purposes.

### FIELD OF THE DISCLOSURE

The present disclosure relates to aerosol generating materials and articles for use in an aerosol provision system, to an aerosol provision system including such materials and articles, and to methods for forming the aerosol generating materials and articles.

### BACKGROUND

Smoking articles such as cigarettes, cigars and the like burn tobacco during use to create tobacco smoke. Alternatives to these types of combustible articles release an inhalable aerosol or vapor by releasing compounds from a substrate material by heating without burning. These may be referred to as non-combustible smoking articles or aerosol generating articles.

One example of such a product is a heating device which release compounds by heating, but not burning, a solid aerosolizable material. This solid aerosolizable material may, in some embodiments, contain a tobacco material. The heating volatilizes at least one component of the material, typically forming an inhalable aerosol. These products may be referred to as heat-not-burn devices, tobacco heating devices or tobacco heating products. Various different arrangements for volatilizing at least one component of the solid aerosolizable material are known.

### BRIEF SUMMARY

The present disclosure is generally directed to an aerosol generating material for use in a non-combustible aerosol provision system. The aerosol generating material comprises an aerosol former, a binder, a non-tobacco botanical material, and an active agent, the active agent comprising at least a substituted 3-(1-methylpyrrolidin-2-yl)pyridine, 3-(azetidin-2-yl)pyridine, or 3-(azetidin-2-ylmethoxy)pyridine. The aerosol-generating material may be incorporated into an article for use in the aerosol-generating system.

The present disclosure includes, without limitation, the following embodiments.

Embodiment 1: An aerosol generating material for use in a non-combustible aerosol provision system, wherein the aerosol generating material is in sheet form, shredded form, bead form, fibrous form, or extrudate form, the aerosol generating material comprising an aerosol former, a binder, a non-tobacco botanical material, and an active agent, the active agent comprising at least a compound having a structure according to Formula I, Formula II, or Formula III:

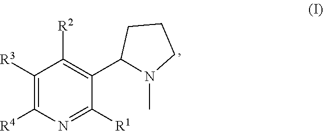

wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and wherein at least one of R¹, R², R³, and R⁴ are not hydrogen; or

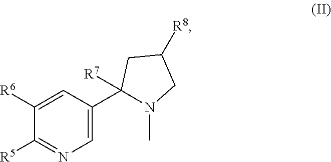

wherein:
   R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
   R⁷ is selected from the group consisting of hydrogen and CH₃;
   R⁸ is selected from the group consisting of hydrogen and C1-C3 alkyl; and
   at least one of R⁷ and R⁸ is not hydrogen; or

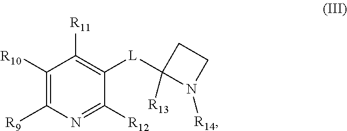

wherein:
   L is a bond or —OCH₂—*, where the asterisk indicates an attachment point to the azetidine ring;
   R⁹, R¹⁰, R¹¹, and R¹² are each independently selected from the group consisting of hydrogen, alkyl, alkoxy, halogen, and cyano;
   R¹³ is H or CH₃; and
   R¹⁴ is H or CH₃.

Embodiment 2: The aerosol generating material of Embodiment 1, wherein the active agent comprises a compound having a structure according to Formula I, and wherein R¹, R², and R³ are each H.

Embodiment 3: The aerosol generating material of Embodiment 1 or 2, wherein the active agent comprises a compound having a structure according to Formula I, and wherein R⁴ is optionally substituted C₁-C₆ alkyl, F, Cl, Br, OCH₃, OEt, or CN.

Embodiment 4: The aerosol generating material of Embodiment 3, wherein R⁴ is C₁-C₃ alkyl, such as wherein R⁴ is CH₃.

Embodiment 5: The aerosol generating material of Embodiment 1, wherein the active agent comprises a compound having a structure according to Formula II, wherein R⁵ and R⁶ are H; R⁷ is CH₃; and R⁸ is H.

Embodiment 6: The aerosol generating material of Embodiment 1, wherein the active agent comprises a compound having a structure according to Formula II, wherein R⁵, R⁶ and R⁷ are H, and R⁸ is CH₃.

Embodiment 7: The aerosol generating material of any one of Embodiments 1-6, wherein the aerosol generating material further comprises a binder and a non-tobacco botanical material, optionally wherein the non-tobacco botanical material comprises fennel, star anise, hemp, rooibos, or a combination thereof.

Embodiment 8: The aerosol generating material of any one of Embodiments 1-7, wherein the aerosol generating material further comprises a filler.

Embodiment 9: The aerosol generating material of any one of Embodiments 1-8, wherein the aerosol generating material is in the form of a sheet or a shredded sheet.

Embodiment 10: The aerosol generating material of any one of Embodiments 1-8, wherein the aerosol generating material is in the form of a plurality of beads.

Embodiment 11: The aerosol generating material of any one of Embodiments 1-8, wherein the aerosol generating material is in an extruded solid form, optionally wherein the extruded solid form comprises one or more passages therethrough to allow air to pass through the extruded solid form, and optionally wherein the extruded solid form has a cavity adapted to receive an aerosol generator, such as a heater.

Embodiment 12: The aerosol generating material of Embodiment 11, further comprising an end plug adjacent to the extruded solid aerosol generating material, optionally wherein the end plug has a cavity adapted to receive an aerosol generator, such as a heater.

Embodiment 13: The aerosol generating material of any one of Embodiments 1-8, wherein the aerosol generating material is in a fibrous form, such as in the form of a nonwoven material, optionally wherein the nonwoven material comprises regenerated cellulose.

Embodiment 14: The aerosol generating material of any one of embodiments 1-13, wherein the active agent further comprises a nicotine component, a cannabinoid, a terpene, caffeine, an amino acid, a vitamin, melatonin, a botanical extract, or a combination thereof.

Embodiment 15: The aerosol generating material of any one of Embodiments 1-14, wherein the active agent further comprises a nicotine component.

Embodiment 16: The aerosol generating material of any one of Embodiments 1-14, wherein the aerosol generating material is substantially free of a nicotine component.

Embodiment 17: The aerosol generating material of any one of Embodiments 1-16, wherein the aerosol generating material further comprises a flavorant.

Embodiment 18: The aerosol generating material of any one of Embodiments 1-17, wherein the aerosol generating material is substantially free of tobacco material.

Embodiment 19: An aerosol provision system comprising the aerosol generating material of any one of Embodiments 1-18.

Embodiment 20: A method of forming an aerosol generating material in sheet form, shredded form, bead form, or extrudate form, the aerosol generating material comprising an aerosol former, a binder, a non-tobacco botanical material, and an active agent, the active agent comprising at least a substituted 3-(1-methylpyrrolidin-2-yl)pyridine, 3-(azetidin-2-yl)pyridine, or 3-(azetidin-2-ylmethoxy)pyridine, the method comprising:
mixing aerosol former, binder, non-tobacco botanical material, and active agent to form a slurry; and
extruding the slurry through a die to form the aerosol generating material.

Embodiment 21: A method of forming an aerosol generating material in sheet form, shredded form, bead form, or extrudate form, the aerosol generating material comprising an aerosol former, a binder, a non-tobacco botanical material, and an active agent, the active agent comprising at least a substituted 3-(1-methylpyrrolidin-2-yl)pyridine, 3-(azetidin-2-yl)pyridine, or 3-(azetidin-2-ylmethoxy)pyridine, the method comprising:
mixing aerosol former, binder, and non-tobacco botanical material to form a slurry;
extruding the slurry through a die to form an extrudate; and
applying the active agent to the extrudate (or a sheet or shredded material formed from the extrudate) to form the aerosol generating material.

Embodiment 22: The aerosol generating material of Embodiment 1, wherein the active agent has a structure according to Formula II, optionally wherein R⁵ is optionally substituted C₁-C₆ alkyl, F, Cl, Br, OCH₃, OCH₂CH₃, or CN; and R⁶ is H, or optionally wherein R⁵ is H; and R⁶ is optionally substituted C₁-C₆ alkyl, F, Cl, Br, OCH₃, OCH₂CH₃, or CN.

Embodiment 23: The aerosol generating material of Embodiment 1, wherein the active agent has a structure according to Formula III, optionally wherein R⁹ is H or CH₃; and R¹⁰, R¹¹, and R¹² are each H.

These and other features, aspects, and advantages of the present disclosure will be apparent from a reading of the following detailed description together with the accompanying figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable, unless the context of the disclosure clearly dictates otherwise.

### BRIEF DESCRIPTION OF THE DRAWINGS

Having thus described aspects of the disclosure in the foregoing general terms, reference will now be made to the accompanying figures, which are not necessarily drawn to scale, and wherein:

FIG. 1 depicts the steps of a process used to manufacture an aerosol generating material according to a non-limiting embodiment of the disclosure.

FIG. 2 is a side-on cross-sectional view of an article comprising the aerosol generating material according to a non-limiting embodiment of the disclosure.

FIG. 3 is a perspective illustration of a non-combustible aerosol provision device for generating aerosol from an article according to a non-limiting embodiment of the disclosure.

FIG. 4 is a cross-sectional view of a non-combustible aerosol provision device according to a non-limiting embodiment of the disclosure.

### DETAILED DESCRIPTION

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements.

As used in this specification and the claims, the singular forms “a,” “an,” and “the” include plural referents unless the context clearly dictates otherwise.

Reference to “dry weight percent” or “dry weight basis” refers to weight on the basis of dry ingredients (i.e., all ingredients except water). All weight percent values herein are dry weight percent unless otherwise indicated.

The terms ‘upstream’ and ‘downstream’ used herein are relative terms defined in relation to the direction of mainstream aerosol drawn though an article or device in use.

Unless otherwise defined herein, by “substantially free” it is meant that the noted component (e.g., nicotine or a tobacco material) has not been intentionally added, beyond trace amounts that may be present e.g., as an impurity in another component, or small amounts which may be present in certain flavor packages. For example, some embodiments can have less than 0.01% by weight of the noted component, or less than 0.001%, or even 0% by weight of the noted component, based on the total weight of the aerosol generating material. In some embodiments, the aerosol generating material is completely free of the noted component (i.e., having 0% or as having an amount below the limit of detection).

### Aerosol Generating Material

The present disclosure is generally directed to an aerosol generating material for use in a non-combustible aerosol provision system and to aerosol generating articles and aerosol provision systems comprising said material. As used herein the term “aerosol generating material” refers to a material that is capable of generating aerosol, for example when heated, irradiated or energized in any other way. An aerosol generating material may be in the form of a solid, liquid or gel. The aerosol generating material of the present disclosure comprises an aerosol former, a binder, a non-tobacco botanical material, and an active agent. Each of these components is further described herein below.

### Aerosol Former

The aerosol generating material as disclosed herein comprises an aerosol former. In this context, an “aerosol former” or “aerosol forming material” is an agent that promotes the generation of an aerosol. An aerosol former may promote the generation of an aerosol by promoting an initial vaporization and/or the condensation of a gas to an inhalable solid and/or liquid aerosol. In general, any suitable aerosol former may be included in the aerosol generating material of the disclosure. The particular choice of aerosol former(s) may depend on factors such as the method of aerosol formation, the appearance and volume of the aerosol, the desired density of the aerosol, and the like. Suitable aerosol formers include, but are not limited to: a polyol such as sorbitol, glycerol, and glycols like propylene glycol or triethylene glycol; a non-polyol such as monohydric alcohols, high boiling point hydrocarbons, acids such as lactic acid, glycerol derivatives, esters such as diacetin, triacetin, triethylene glycol diacetate, triethyl citrate or myristates including ethyl myristate and isopropyl myristate and aliphatic carboxylic acid esters such as methyl stearate, dimethyl dodecanedioate and dimethyl tetradecanedioate. In some embodiments, the aerosol former may be glycerol, propylene glycol, or a mixture of glycerol and propylene glycol.

In some embodiments, the aerosol former may comprise one or more of glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

In some embodiments, the aerosol former comprises one or more compound selected from erythritol, propylene glycol, glycerol, triacetin, sorbitol and xylitol. In some embodiments, the aerosol former material comprises, consists essentially of, or consists of, glycerol.

In some embodiments, the aerosol former comprises a mixture of glycerol and propylene glycol in a weight ratio of glycerol to propylene glycol of from about 3:1 to about 1:3, from about 2:1 to about 1:2, from about 1.5:1 to about 1:1.5, from about 55:45 to about 45:55, or about 45:55. In some embodiments, the aerosol generating material is substantially free or completely free of propylene glycol. In some embodiments, the only aerosol former present is glycerol.

The total amount of aerosol former in the aerosol generating material may vary. In some embodiments, the total amount of aerosol former is at least about 5% by weight or at least about 7.5% by weight or at least about 10% by weight, based on the total weight of aerosol generating material. In some embodiments, the aerosol former is present in an amount of about 5% by weight to about 40% by weight, such as about 10% to about 25% by weight.

### Binder

The aerosol generating material as disclosed herein comprises a binder. The binder generally comprises an alginate, pectin, starch (and derivatives), cellulose (and derivatives), natural gums, silica or silicone compounds, clay, polyvinyl alcohol, or combinations thereof. For example, in some embodiments, the binder comprises one or more of alginates, pectins, hydroxyethyl cellulose, hydroxypropyl cellulose, carboxymethylcellulose (CMC), pullulan, xanthan gum, guar gum, carrageenan, agarose, acacia gum, curdlan gum, fumed silica, PDMS, sodium silicate, kaolin and polyvinyl alcohol. In some embodiments, the binder comprises alginate and/or pectin or carrageenan. In some embodiments, the binder comprises CMC.

The total amount of binder in the aerosol generating material may vary. In some embodiments, the total amount of binder is at least about 1% by weight or at least about 2% by weight or at least about 3% by weight, based on the total weight of aerosol generating material. In some embodiments, the total amount of binder is present in an amount of about 1% by weight to about 20% by weight, such as about 2% to about 15% by weight.

### Non-Tobacco Botanical Material

The aerosol generating material as disclosed herein comprises a non-tobacco botanical material. As used herein, the term “botanical” includes any material derived from plants including, but not limited to, extracts, leaves, bark, fibers, stems, roots, seeds, flowers, fruits, pollen, husk, shells or the like. A “non-tobacco botanical material” is a botanical material other than tobacco. The use of a non-tobacco botanical material may enhance the sensorial properties of an aerosol produced by the aerosol generating material. The material may be in the form of liquid, gas, solid, powder, dust, crushed particles, granules, pellets, shreds, strips, sheets, or the like. In some embodiments, the botanical material is in the form of a solid. In some embodiments, the botanical material comprises leaf material. The botanical material may be a particulate or granular material. In some embodiments, the non-tobacco botanical material is a powder. The non-tobacco botanical material may be formed by grinding solid non-tobacco botanical material to produce a ground non-tobacco botanical material. Alternatively or in addition, the non-tobacco botanical material may comprise strips, strands or fibers of non-tobacco botanical material. For example, the non-tobacco botanical material may comprise particles, granules, fibers, strips and/or strands of non-tobacco botanical material. In some embodiments, the non-tobacco botanical material consists of particles or granules of non-tobacco botanical material. In some embodiments, the non-tobacco botanical material is in a particulate or ground form.

Example non-tobacco botanical material include, but are not limited to, eucalyptus, star anise, hemp, cocoa, cannabis, fennel, lemongrass, peppermint, spearmint, Aspalathus linearis (rooibos; red or green), chamomile, flax, ginger, Ginkgo biloba, hazel, hibiscus, laurel, licorice (liquorice), matcha, mate, orange skin, papaya, rose, sage, tea such as green tea or black tea, thyme, clove, cinnamon, coffee, aniseed (anise), basil, bay leaves, cardamom, coriander, cumin, nutmeg, oregano, paprika, rosemary, saffron, lavender, lemon peel, mint, juniper, elderflower, vanilla, wintergreen, beefsteak plant, curcuma, turmeric, sandalwood, cilantro, bergamot, orange blossom, myrtle, cassis, valerian, pimento, mace, damien, marjoram, olive, lemon balm, lemon basil, chive, carvi, verbena, tarragon, geranium, mulberry, ginseng, theanine, theacrine, maca, ashwagandha, damiana, guarana, chlorophyll, baobab or any combination thereof. The mint may be chosen from the following mint varieties: Mentha Arventis, Mentha c.v., Mentha niliaca, Mentha piperita, Mentha piperita citrata c.v., Mentha piperita c.v, Mentha spicata crispa, Mentha cardifolia, Mentha longifolia, Mentha suaveolens variegata, Mentha pulegium, Mentha spicata c.v. and Mentha suaveolens. In some embodiments, the non-tobacco botanical material is selected from the group consisting of fennel, star anise, rooibos and mixtures thereof. In some embodiments, the non-tobacco botanical material comprises or is rooibos.

In embodiments in which the non-tobacco botanical material is a particulate non-tobacco botanical material, each particle of the material may have a maximum dimension. As used herein, the term “maximum dimension” refers to the longest straight-line distance from any point on the surface of a particle of material, or on a particle surface, to any other surface point on the same particle of material, or particle surface. The maximum dimension of a particle of particulate material may be measured using scanning electron microscopy (SEM).

In some embodiments, the maximum dimension of each particle of non-tobacco botanical material is up to about 800 μm. In some embodiments, the maximum dimension of each particle of non-tobacco botanical material is up to about 2000 μm. In some embodiments, the maximum dimension of each particle of non-tobacco botanical material is about 200 μm to about 800 μm.

A population of particles of the non-tobacco botanical material may have a particle size distribution (D90) of at least about 100 μm. In some embodiments, a population of particles of the non-tobacco botanical material has a particle size distribution (D90) of at least about 50 μm, of at least about 60, of at least about 70 μm, of at least about 80 μm, of at least about 90, of at least about 100 μm, of at least about 110 μm, of at least about 120 μm, of at least about 130 μm. In some embodiments, a population of particles of the non-tobacco botanical material has a particle size distribution (D90) of at most about 720 μm, of at most about 740 μm, of at most about 760 μm, of at most about 780 μm, of at most about 800 μm, of at most about 820 μm, of at most about 840 μm, of at most about 860 μm. In some embodiments, a population of particles of the non-tobacco botanical material has a particle size distribution (D90) of about 600 μm. A particle size and shape analyzer, such as a Camsizer may be used to measure the particle size distribution, and sieve analysis may be used to determine the particle size distribution of the particles of non-tobacco botanical material.

The particle size distribution (D90) of the non-tobacco botanical material may be controlled to achieve the desired area density of the aerosol generating material. The area density of the material may be measured in GSM (grams per square meter or g/m²). For example, lower particle size distributions (D90) are associated with higher area densities. When the aerosol generating material is incorporated into an article for use in a non-combustible aerosol provision system, this higher area density may decrease the fill value of the non-tobacco botanical material. A particular example of this is that a particle size distribution (D90) of 320 is predicted to provide an area density of around 190 to 200 g/m².

A lower area density may be associated with better taste and sensory properties of the aerosol generating material. Without wishing to be bound by theory, the superior taste and sensory properties may be attributable to improved heat transfer through the material. It is thought that the lower area density facilitates heat transfer through the aerosol generating material, which facilitates aerosol generation and thus improves the sensory characteristics of the material. As the process for producing the aerosol generating material produced by the process of the present disclosure involves less drying than conventional processes, fewer volatile components, many of which are considered to be desirable are lost. The taste and aroma is better retained and so this is associated with better sensory properties. In addition, the process requires less energy to remove volatile compounds.

The particle size distribution (D90) may be controlled to achieve the desired tensile strength of the aerosol generating material. For example, higher particle size distributions (D90) are associated with lower tensile strengths. Without wishing to be bound by theory, when the particle size distribution is higher, there is less material to be bound together. This may make the aerosol generating material weaker and so the tensile strength is lower. A particle size distribution (D90) of 160 to 450 μm, in particular 300 to 350 μm, may provide an optimal tensile strength.

A balance may be achieved between the optimum area density and the tensile strength of the aerosol generating material and this balance can be achieved by selection of the particle size distribution (D90). The particle size distribution (D90) should be low enough to provide an adequate tensile strength but be high enough to provide an area density that provides positive sensory properties for the user and provides easier removal of volatile compounds. For example, the particle size distribution (D90) may be selected to provide a low enough area density to provide positive sensory properties, but a high enough tensile strength to be within the operational limits of the manufacturing machinery.

A particle size distribution (D90) of at least about 100 μm is thought to contribute to the tensile strength of the aerosol generating material. In some embodiments, a particle size distribution (D90) of less than 100 μm has been found according to the present disclosure to provide an aerosol generating material which has a good tensile strength. However, the inclusion of such fine particles in the aerosol generating material can increase its density. When the aerosol generating material is incorporated into an article for use in a non-combustible aerosol provision system, this higher density may decrease the fill value of the material. In some embodiments, the particle size distribution (D90) is 100-800 μm. In some embodiments, the particle size distribution (D90) is 160-450 μm. In some embodiments, the particle size distribution (D90) is 200-450 μm.

Selection of the particle size distribution (D90) can provide an adequate tensile strength and area density of the aerosol-generating material that are the same or an improvement on aerosol-generating materials that are produced via “bandcasting” techniques.

The total amount of non-tobacco botanical material in the aerosol generating material may vary. In some embodiments, the total amount of non-tobacco botanical material is at least about 10% by weight or at least about 20% by weight or at least about 30% by weight, based on the total weight of aerosol generating material. In some embodiments, the non-tobacco botanical material is present in an amount of about 10% by weight to about 80% by weight, such as about 30% to about 70% by weight.

In some embodiments, all or a portion of the non-tobacco botanical material can be replaced by tobacco material.

### Active Agent

### Substituted 3-(1-methylpyrrolidin-2-yl)pyridine

In some embodiments, the aerosol generating material of the disclosure comprises an active agent, including, but not limited to, a substituted 3-(1-methylpyrrolidin-2-yl)pyridine. The active agent may further include components such as cannabinoids, terpenes, vitamins, melatonin, caffeine, tobacco extract, or combinations thereof. The various components are described further herein below.

As used herein, the term “substituted 3-(1-methylpyrrolidin-2-yl)pyridine” refers to a compound having a 3-(1-pyrrolidin-2-yl)pyridine) scaffold and bearing one or more non-hydrogen substituents on the pyridine ring.

In some embodiments, the substituted 3-(1-methylpyrrolidin-2-yl)pyridine has a structure according to Formula I:

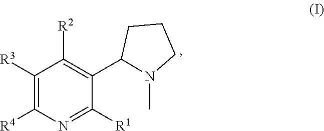

wherein:
R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and at least one of R¹, R², R³, and R⁴ are not hydrogen.

Substituted 3-(1-methylpyrrolidin-2-yl)pyridines with various R¹, R², R³, and R⁴ substituents have been reported previously. See for example, U.S. Pat. Nos. 4,321,387, 4,155,909; 5,015,741, 5,138,062, and 5,703,100, each of which is incorporated by reference herein and describe such substituted 3-(1-methylpyrrolidin-2-yl)pyridines, their synthesis, and pharmacological properties. Substituted 3-(1-methylpyrrolidin-2-yl)pyridines and their pharmacological profiles have also been disclosed in Wang et al., Drug Development Research 1998, Volume 45, Issue 1, Pages 10-16; and Dukat et al. European Journal of Medicinal Chemistry 1999, 34(1): 31-40.

In some embodiments, R¹, R², and R⁴ are each H, and R³ is a non-hydrogen substituent. In some embodiments, R³ is optionally substituted C₁-C₆ alkyl, F, Cl, Br, OMe, OEt, or CN. In some embodiments, R³ is Me or Et. n some embodiments, R³ is F.

In some embodiments, R¹, R², and R³ are each H, and R⁴ is a non-hydrogen substituent.

In some embodiments, R⁴ is optionally substituted C₁-C₆ alkyl, F, Cl, Br, OMe, OEt, or CN.

In some embodiments, R¹, R², and R³ are each H, and R⁴ is optionally substituted C₁-C₆ alkyl, F, Cl, Br, OCH₃, OEt, or CN.

In some embodiments, R¹, R², and R³ are each H, and R⁴ is C₁-C₃ alkyl.

In some embodiments, R¹, R², and R³ are each H, and R⁴ is Me. In such embodiments, the compound of Formula I may be referred to as 2-methyl-5-(1-methylpyrrolidin-2-yl)pyridine.

The pharmacology of 2-methyl-5-(1-methylpyrrolidin-2-yl)pyridine has been reported in, for example, Dukat et al. European Journal of Medicinal Chemistry, Volume 31, Issue 11, 1996, Pages 875-888 (incorporated herein by reference), and the pharmacological profile of the (S)-enantiomer of 2-methyl-5-(1-methylpyrrolidin-2-yl)pyridine in the form of the benzoate salt (CAS 2861225-70-7; referred to as Imotine™) is discussed in Carmines et al, Poster #6; 76^{th} TSRC Conference 2023, Norfolk, VA, USA).

In some embodiments, the substituted 3-(1-methylpyrrolidin-2-yl)pyridine has a structure according to Formula II:

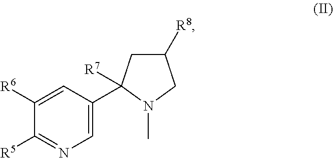

wherein:
   R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
   R⁷ is selected from the group consisting of hydrogen and CH₃;
   R⁸ is selected from the group consisting of hydrogen and C1-C3 alkyl; and
   at least one of R⁷ and R⁸ is not hydrogen.

Certain substituted 3-(1-methylpyrrolidin-2-yl)pyridines with various R⁵, R⁶, R⁷, and R⁸ substituents have been reported previously. See for example, U.S. Pat. Nos. 4,321,387, 4,155,909; 5,015,741, 5,138,062, and 5,703,100, each of which is incorporated by reference herein and describe example substituted 3-(1-methylpyrrolidin-2-yl)pyridines, their synthesis, and pharmacological properties. Certain substituted 3-(1-methylpyrrolidin-2-yl)pyridines and their pharmacological profiles have also been disclosed in Wang et al., Drug Development Research 1998, Volume 45, Issue 1, Pages 10-16; Dukat et al. European Journal of Medicinal Chemistry 1999, 34(1): 31-40; Lin et al., J. Med. Chem. (1994), 37, 3542-3553.

In some embodiments, R⁵ and R⁶ are H; R⁷ is CH₃; and R⁸ is H. In such embodiments, the compound of Formula II may be referred to as 3-(1,2-dimethylpyrrolidin-2-yl)pyridine, or 2′-methyl-5-(1-methylpyrrolidin-2-yl)pyridine, and has a structure:

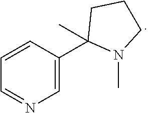

The compound 3-(1,2-dimethylpyrrolidin-2-yl)pyridine is known in the literature, and has a Chemical Abstracts Registry Number of 220650-38-4. The synthesis of this compound has been reported in Rouchaud et al., J Het Chem 2012, 49(1), 161-166; Wang et al., Drug Dev Res 1998, 45(1), 10-16; Secor et al., Tetrahedron Lett. (1981), 22(33), 3151-3154; US Patent Application Publication No. 2013/0157995; PCT Application Publication No. WO2012/031220; and U.S. Pat. No. 9,440,948, each of which are incorporated by reference herein with respect to the synthesis of 3-(1,2-dimethylpyrrolidin-2-yl)pyridine.

In some embodiments, R⁵ and R⁶ are H; R⁷ is H; and R⁸ is CH₃. In such embodiments, the compound of Formula II may be referred to as 3-(1,4-dimethylpyrrolidin-2-yl)pyridine, or 4′-methyl-5-(1-methylpyrrolidin-2-yl)pyridine, and has a structure:

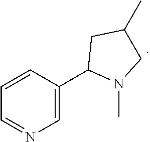

The compound 3-(1,4-dimethylpyrrolidin-2-yl)pyridine is known in the literature, has a Chemical Abstracts Registry Number of 74805-00-8, and is commercially available from, for example, Enamine Stock Building Blocks and Aurora Building Blocks. The synthesis of this compound has been reported in U.S. Pat. No. 9,440,948; and EP Patent No. 559495, each of which are incorporated by reference herein with respect to the synthesis of 3-(1,4-dimethylpyrrolidin-2-yl)pyridine.

In some embodiments, R⁵ is CH₃; R⁶ is H; R⁷ is CH₃; and R⁸ is H. In such embodiments, the compound of Formula II may be referred to as 5-(1,2-dimethylpyrrolidin-2-yl)-2-methylpyridine, and has a structure:

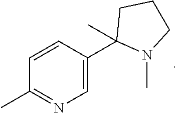

The compound 5-(1,2-dimethylpyrrolidin-2-yl)-2-methylpyridine may be readily synthesized according to known reactions. For example, commercially available 2-methyl-5-(2-methylpyrrolidin-2-yl)pyridine (Chemical Abstracts Registry Number of 1528955-30-7; Aurora Building Blocks, Adlab Chemicals Building Blocks) can be N-methylated with formaldehyde and formic acid, or alternatively with formaldehyde and a reducing agent such as sodium cyanoborohydride to afford 5-(1,2-dimethylpyrrolidin-2-yl)-2-methylpyridine. Alternatively, 5-(1,2-dimethylpyrrolidin-2-yl)-2-methylpyridine may be synthesized by lithiation of 2-methyl-5-bromopyridine, reaction of the lithiated pyridine with N-methylpyrrolidone, and addition of methyl lithium to the perchlorate salt of the resulting imine. This reaction sequence is shown below in Scheme 1.

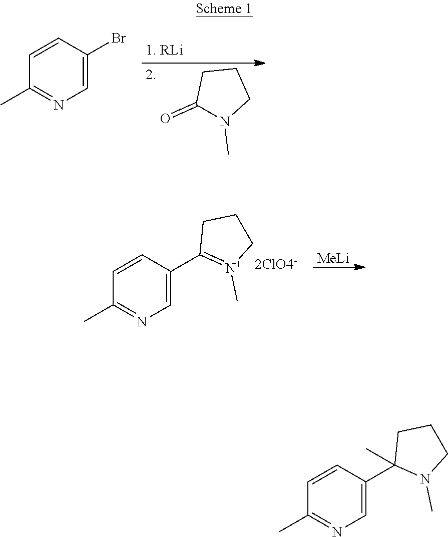

In some embodiments, R⁵ is H or CH₃, R⁶ is H, R⁷ is H or CH₃, and R¹ is H or CH₃, provided that at least one of R⁷ and R⁸ is CH₃. Accordingly, in some embodiments, the substituted 3-(1-methylpyrrolidin-2-yl)pyridine has a structure selected from:

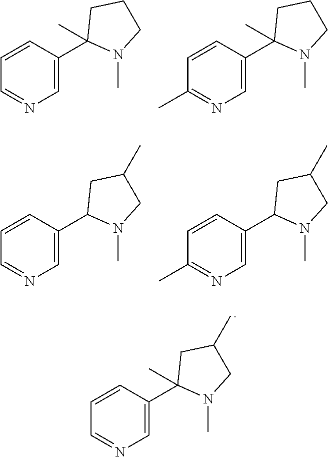

In some embodiments, R⁵ is H, R⁶ is F, CH₃, or OCH₃, R⁷ is H or CH₃, and R⁸ is H or CH₃, provided that at least one of R⁷ and R⁸ is CH₃. Accordingly, in some embodiments, the substituted 3-(1-methylpyrrolidin-2-yl)pyridine has a structure selected from:

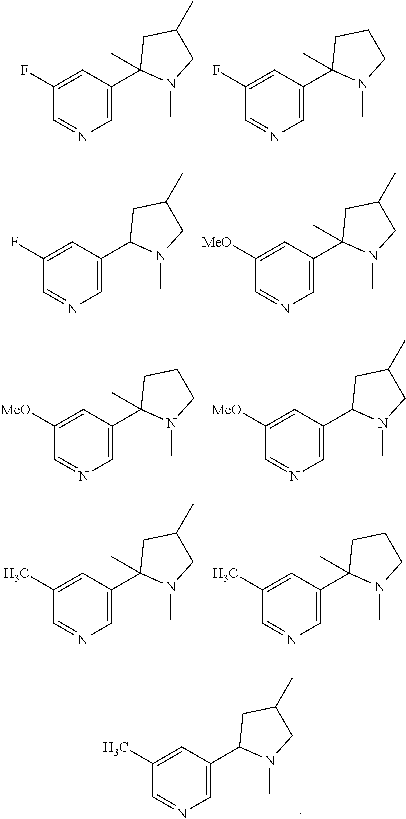

A substituted 3-(1-methylpyrrolidin-2-yl)pyridine and bearing one or more substituents on the pyrrolidine ring as described herein may be present as a single enantiomer or as a mixture of enantiomers. In some embodiments, the substituted 3-(1-methylpyrrolidin-2-yl)pyridine bearing one or more substituents on the pyrrolidine ring is present in racemic form, meaning there are equal amounts of (R)- and (S)-enantiomers present. In some embodiments, the aerosol generating material comprises unequal amounts of (R)- and (S)-enantiomer (i.e., is enriched in either the (R)- or (S)-enantiomer. In some embodiments, the aerosol generating material predominantly comprises the substituted 3-(1-methylpyrrolidin-2-yl)pyridine bearing one or more substituents on the pyrrolidine ring in the (R)-configuration, for example, about 90% or more of the total quantity of substituted 3-(1-methylpyrrolidin-2-yl)pyridine bearing one or more substituents on the pyrrolidine ring present is in the (R)-configuration. In some embodiments, the aerosol generating material predominantly comprises the substituted 3-(1-methylpyrrolidin-2-yl)pyridine bearing one or more substituents on the pyrrolidine ring in the (S)-configuration, for example, about 90% or more of the total quantity of substituted 3-(1-methylpyrrolidin-2-yl)pyridine present is in the (S)-configuration. In some embodiments, the aerosol generating material comprises 95% or more of the (S)-configuration of the substituted 3-(1-methylpyrrolidin-2-yl)pyridine, based on the total amount of substituted 3-(1-methylpyrrolidin-2-yl)pyridine present.

In some embodiments, the substituted 3-(1-methylpyrrolidin-2-yl)pyridine bearing one or more substituents on the pyrrolidine ring is non-racemic, and has one of the following structures:

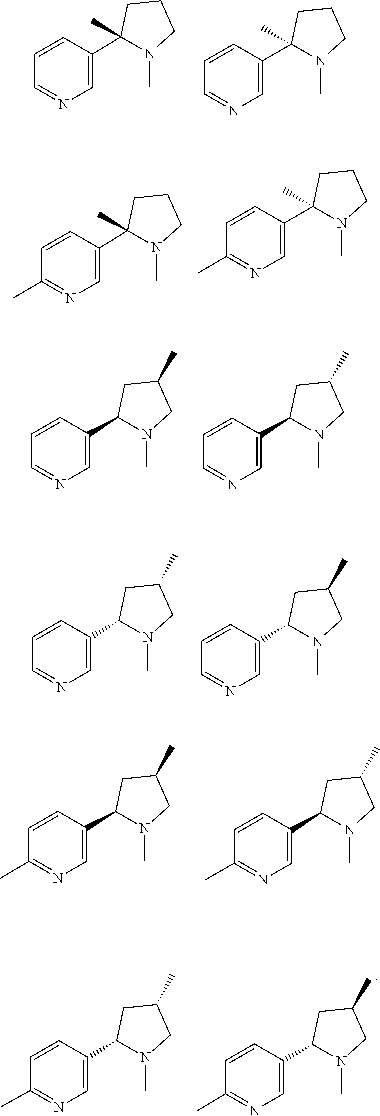

In some embodiments, the substituted 3-(1-methylpyrrolidin-2-yl)pyridine bearing one or more substituents on the pyrrolidine ring is non-racemic, and has a structure selected from:

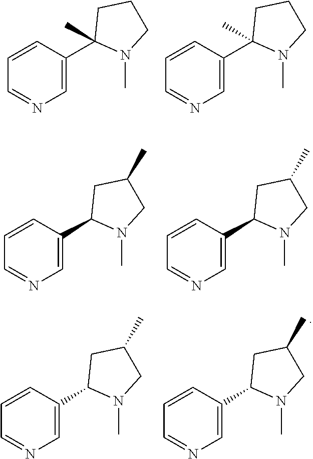

Such single enantiomer or enantiomerically enriched compounds may be obtained through classical resolution techniques using salt formation with chiral acids to form diastereomeric salts separable by crystallization. Suitable chiral acids include, but are not limited to, (R)- or (S)-dibenzoyl tartaric acid, di-p-toluoyl tartaric acid, or di-p-anisolyl tartaric acid; (R)- or (S)-mandelic acid, and (R)- or (S)-10-camphorsulfonic acid. Alternatively, one of skill in the art will recognize opportunities for chiral syntheses using either commercially available starting materials with established chiral centers or through the use of chiral auxiliary chemistries. For example, preparation of the 2S,4R enantiomer of 3-(1,4-dimethylpyrrolidin-2-yl)pyridine has been reported in, for example, U.S. Pat. No. 4,332,945, incorporated herein by reference with respect to syntheses of chiral nicotine analogs.

The pharmacology of various substituted 3-(1-methylpyrrolidin-2-yl)pyridines such as those described herein has been reported in, for example, Lin et al., J. Med. Chem., 1994, 37, 3542-3553; Dukat et al. European Journal of Medicinal Chemistry, 31(11), 1996, 875-888; U.S. Pat. No. 9,440,948; Wang et al., Drug Dev Res 1998, 45(1), 10-16 (each of which is incorporated herein by reference), among many others. Generally, small substituents such as methyl groups are well tolerated at the 2′ or 4′ positions of the nicotine pyrrolidine ring, and small substituents such as alkyl, halogen, alkoxy, and the like are well tolerated at the 5 or 6 position of the nicotine pyridine ring. For example, 3-(1-methylpyrrolidin-2-yl)pyridines bearing a methyl substituent at the 2′ or 4′ position are equipotent or even more potent than nicotine with respect to binding affinity to the nicotinic acetylcholine receptor, and are expected to preserve the pharmacological effects of nicotine in vivo. See, for example U.S. Pat. No. 5,278,176, Lin et al., J. Med. Chem., 1994, 37, 3542-3553, and Wang et al., Drug Dev Res 1998, 45(1), 10-16.

Without wishing to be bound by any particular theory, it is believed that certain substitutions for hydrogen on the pyridine ring and/or pyrrolidine rings of the substituted 3-(1-methylpyrrolidin-2-yl)pyridine compound preserve the general pharmacological profile and physiological effects of nicotine while offering the potential for one or more of greater potency, longer half-life, reduced product consumption, more rapid and/or complete absorption, greater bioavailability, and the like. Particularly, it is believed that in some embodiments, substituted 3-(1-methylpyrrolidin-2-yl)pyridines of the disclosure are readily absorbed through membranes such as pulmonary alveoli, oral mucosa, and the like by virtue of their lipophilicity. Lipophilicity is conveniently measured in terms of log P, the partition coefficient of a molecule between a lipophilic phase and an aqueous phase, usually octanol and water, respectively. Accordingly, in some embodiments, the substituted 3-(1-methylpyrrolidin-2-yl)pyridine of Formula I or II has a calculated or experimental log P of about 1 or greater, where log P is the log₁₀ of the partitioning coefficient of the substituted 3-(1-methylpyrrolidin-2-yl)pyridine between octanol and water. Log P values may be measured experimentally according to protocols well known to one of skill in the art. Alternatively, log P values may be calculated using commercially available software. In some embodiments, the substituted 3-(1-methylpyrrolidin-2-yl)pyridine of Formula I or II has a calculated log P from 1 to about 2, such as 1.1, 1.2, 1.3, 1.4, 1.5, 1.6, 1.7, 1.8, or 1.9. In some embodiments, the substituted 3-(1-methylpyrrolidin-2-yl)pyridine of Formula I or II has a calculated log P from about 1.2 to about 1.7. In some embodiments, the substituted 3-(1-methylpyrrolidin-2-yl)pyridine having a calculated log P from about 1.2 to about 1.7 bears one or more lipophilic substituents on the pyridine ring, such as C₁-C₆ alkyl, or C₁-C₃ alkyl. In some embodiments, the lipophilic substituent is methyl, and is present at the 2, 4, 5, or 6 position of the pyridine ring. In some embodiments, the lipophilic substituted 3-(1-methylpyrrolidin-2-yl)pyridine is 2-methyl-5-(1-methylpyrrolidin-2-yl)pyridine.

The quantity of substituted 3-(1-methylpyrrolidin-2-yl)pyridine (e.g., 2-methyl-5-(1-methylpyrrolidin-2-yl)pyridine) present in the aerosol generating material may vary. Typically, the substituted 3-(1-methylpyrrolidin-2-yl)pyridine (e.g., 2-methyl-5-(1-methylpyrrolidin-2-yl)pyridine, calculated as the free base) is present in a concentration of at least about 0.001% by weight of the aerosol generating material, such as in a range from about 0.01% to about 10%. In some embodiments, the substituted 3-(1-methylpyrrolidin-2-yl)pyridine (e.g., 2-methyl-5-(1-methylpyrrolidin-2-yl)pyridine) is present in a concentration from about 0.1% w/w to about 10% by weight, such as, e.g., from about from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the aerosol generating material. In some embodiments, the substituted 3-(1-methylpyrrolidin-2-yl)pyridine (e.g., 2-methyl-5-(1-methylpyrrolidin-2-yl)pyridine) is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the aerosol generating material. In some embodiments, the aerosol generating material comprises 2-methyl-5-(1-methylpyrrolidin-2-yl)pyridine in an amount from about 0.01 to about 1% by weight, such as from about 0.1 to about 0.75% by weight, based on the total weight of the aerosol generating material.

The substituted 3-(1-methylpyrrolidin-2-yl)pyridine (e.g., 2-methyl-5-(1-methylpyrrolidin-2-yl)pyridine) may be present in the aerosol generating material as the free base, as a salt with a suitable acid, in the form of an ion pair with an organic acid, or a combination thereof. Each of these forms is described further herein below.

As described above, the aerosol generating materials of the disclosure do not contain nicotine and do not contain any compounds obtained by chemical reactions utilizing nicotine as a starting material.

### 3-(azetidin-2-yl)pyridines and 3-(azetidin-2-ylmethoxy)pyridines

In some embodiments, the aerosol generating material of the disclosure comprises as an active agent an optionally substituted 3-(azetidin-2-yl)pyridine or an optionally substituted 3-(azetidin-2-ylmethoxy)pyridine. As used herein, the term “substituted 3-(azetidin-2-yl)pyridine” refers to a compound having a 3-(azetidin-2-yl)pyridine scaffold and bearing one or more non-hydrogen substituents on the azetidine ring, and optionally on the pyridine ring. As used herein, the term “substituted 3-(azetidin-2-ylmethoxy)pyridine” refers to a compound having a 3-(azetidin-2-ylmethoxy)pyridine scaffold and bearing one or more non-hydrogen substituents on the azetidine ring, and optionally on the pyridine ring.

In some embodiments, the 3-(azetidin-2-yl)pyridine or 3-(azetidin-2-ylmethoxy)pyridine has a structure according to Formula III:

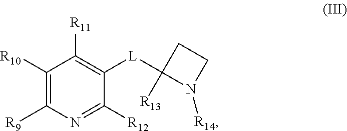

wherein:
L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.

In some embodiments, L is a bond.

In some embodiments, R⁹ is CH₃, F, Cl, Br, OCH₃, OEt, or CN.

In some embodiments, R⁹ is H or CH₃; and R¹⁰, R¹¹, and R¹² are each H.

In some embodiments:
R¹³ and R¹⁴ are both H;
R¹³ and R¹⁴ are both CH₃;
R¹³ is H and R¹⁴ is CH₃; or
R¹³ is CH₃ and R¹⁴ is H.

In some embodiments, the 3-(azetidin-2-yl) pyridine is 3-(azetidin-2-yl)pyridine, and has a structure:

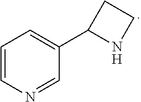

The compound 3-(azetidin-2-yl)pyridine is known in the literature. The synthesis of this compound has been reported in JOC 1979, 44(18), 3136; Med Chem Res (1993) 2:552-5633; in International Patent Application Publication No. WO2012/031220, and in U.S. Pat. Nos. 4,163,855 and 4,163,856, all of which are incorporated herein in their entireties.

In some embodiments, the 3-(azetidin-2-yl) pyridine is 3-(1-methylazetidin-2-yl)pyridine, having the structure:

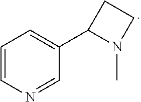

The compound 3-(1-methylazetidin-2-yl)pyridine is known in the literature. The synthesis of this compound has been reported in International Patent Application Publication No. WO2012/031220, previously incorporated by reference herein.

In some embodiments, the 3-(azetidin-2-yl) pyridine has a structure selected from the group consisting of:

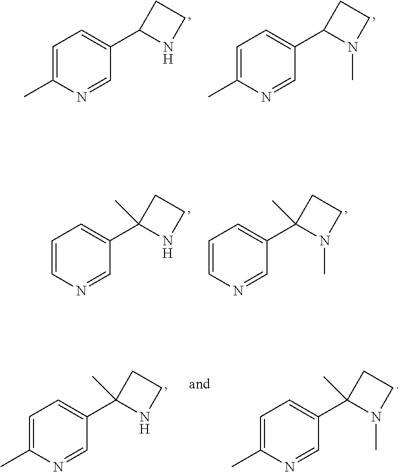

Such compounds are either known, or may be readily prepared according to adaptations of methods utilized for preparation of related 3-(azetidin-2-yl) pyridines and 3-(1-methylpyrrolidin-2-yl)pyridines described herein above. See, e.g., U.S. Pat. No. 4,163,855, previously incorporated by reference herein. The compound 5-(2-azetidinyl)-2-methylpyridine is known in the literature and has a Chemical Abstracts Registry (CAS) Number of 1270467-65-6, and the R- and S-enantiomers have CAS numbers 1213081-15-2 and 1212969-96-4, respectively.

In some embodiments, the active agent comprises a 3-(azetidin-2-ylmethoxy)pyridine (i.e., L is —OCH₂—*).

In some embodiments, R⁹ is CH₃, F, Cl, Br, OCH₃, OEt, or CN.

In some embodiments, R⁹ is H or CH₃; and R¹⁰, R¹¹, and R¹² are each H.

In some embodiments:
R¹³ and R¹⁴ are both H;
R¹³ and R¹⁴ are both CH₃;
R¹³ is H and R¹⁴ is CH₃; or
R¹³ is CH₃ and R¹⁴ is H.

In some embodiments, the 3-(azetidin-2-ylmethoxy)pyridine has a structure selected from the group consisting of:

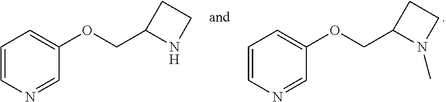

These compounds are known in the literature. The synthesis of these compounds has been reported in International Patent Application Publication No. WO2012/031220, previously incorporated by reference herein.

In some embodiments, the 3-(azetidin-2-ylmethoxy)pyridine has a structure selected from the group consisting of:

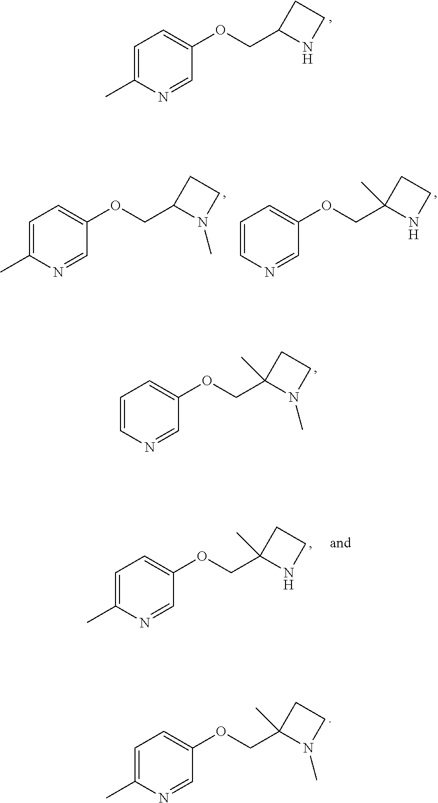

Such compounds are either known, or may be readily prepared according to adaptations of methods utilized for preparation of related the 3-(azetidin-2-ylmethoxy)pyridine, and/or the 3-(azetidin-2-yl)pyridines and substituted 3-(1-methylpyrrolidin-2-yl)pyridines described herein above.

An optionally substituted 3-(azetidin-2-yl)pyridine or optionally substituted 3-(azetidin-2-ylmethoxy)pyridine as described herein may be present as a single enantiomer or as a mixture of enantiomers. In some embodiments, the optionally substituted 3-(azetidin-2-yl)pyridine or optionally substituted 3-(azetidin-2-ylmethoxy)pyridine is present in racemic form, meaning there are equal amounts of (R)- and (S)-enantiomers present. In some embodiments, the aerosol generating material comprises unequal amounts of (R)- and (S)-enantiomer (i.e., is enriched in either the (R)- or (S)-enantiomer). In some embodiments, the aerosol generating material predominantly comprises the optionally substituted 3-(azetidin-2-yl)pyridine or optionally substituted 3-(azetidin-2-ylmethoxy)pyridine in the (R)-configuration, for example, about 90% or more of the total quantity of optionally substituted 3-(azetidin-2-yl)pyridine or optionally substituted 3-(azetidin-2-ylmethoxy)pyridine present is in the (R)-configuration. In some embodiments, the aerosol generating material predominantly comprises the optionally substituted 3-(azetidin-2-yl)pyridine or optionally substituted 3-(azetidin-2-ylmethoxy)pyridine in the (S)-configuration, for example, about 90% or more of the total quantity of optionally substituted 3-(azetidin-2-yl)pyridine or optionally substituted 3-(azetidin-2-ylmethoxy)pyridine present is in the (S)-configuration. In some embodiments, the aerosol generating material comprises 95% or more of the (S)-configuration of the optionally substituted 3-(azetidin-2-yl)pyridine or optionally substituted 3-(azetidin-2-ylmethoxy)pyridine, based on the total amount of optionally substituted 3-(azetidin-2-yl)pyridine or optionally substituted 3-(azetidin-2-ylmethoxy)pyridine present.

In some embodiments, the optionally substituted 3-(azetidin-2-yl)pyridine is non-racemic, and has one of the following structures:

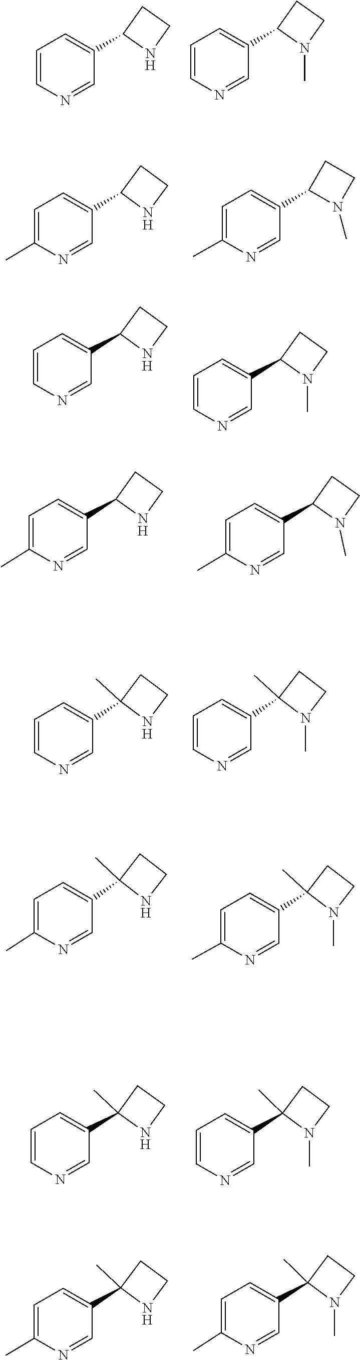

In some embodiments, the optionally substituted 3-(azetidin-2-ylmethoxy)pyridine is non-racemic, and has one of the following structures:

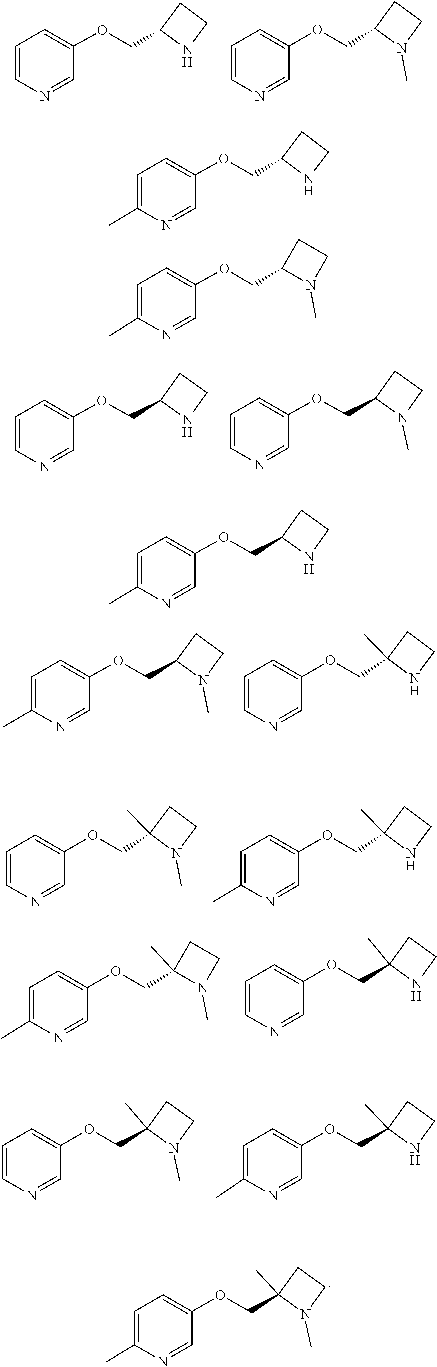

Such single enantiomer or enantiomerically enriched compounds may be obtained through classical resolution techniques using salt formation with chiral acids to form diastereomeric salts separable by crystallization. Suitable chiral acids include, but are not limited to, (R)- or (S)-dibenzoyl tartaric acid, di-p-toluoyl tartaric acid, or di-p-anisolyl tartaric acid; (R)- or (S)-mandelic acid, and (R)- or (S)-10-camphorsulfonic acid. Alternatively, one of skill in the art will recognize opportunities for chiral syntheses using either commercially available starting materials with established chiral centers or through the use of chiral auxiliary chemistries. For example, preparation of the 2S,4R enantiomer of 3-(1,4-dimethylpyrrolidin-2-yl)pyridine has been reported in, for example, U.S. Pat. No. 4,332,945, incorporated herein by reference with respect to syntheses of chiral nicotine analogs.

The pharmacology of certain 3-(azetidin-2-yl)pyridines and 3-(azetidin-2-ylmethoxy)pyridines has been previously disclosed, for example, in the references cited herein with respect to synthesis of such compounds. Generally, these compounds exhibit high affinity for one or more subtypes of nicotinic acetylcholine receptors, particularly the a4b2 subtype. The overall pharmacological profiles have been shown to be or are expected to be comparable to that of nicotine.

The quantity of optionally substituted 3-(azetidin-2-yl)pyridine or 3-(azetidin-2-ylmethoxy)pyridine present in the aerosol generating material may vary. Typically, the optionally substituted 3-(azetidin-2-yl)pyridine or 3-(azetidin-2-ylmethoxy)pyridine, calculated as the free base, is present in a concentration of at least about 0.001% by weight of the aerosol generating material, such as in a range from about 0.01% to about 10%. In some embodiments, the optionally substituted 3-(azetidin-2-yl)pyridine or 3-(azetidin-2-ylmethoxy)pyridine is present in a concentration from about 0.05% w/w to about 5% by weight, such as, e.g., from about from about 0.05% w/w. about 0.1% w/w, about 0.2%, about 0.3%, about 0.4%, about 0.5% about 0.6%, about 0.7%, about 0.8%, or about 0.9%, to about 1%, about 2%, about 3%, about 4%, or about 5% by weight, calculated as the free base and based on the total weight of the aerosol generating material. In some embodiments, the optionally substituted 3-(azetidin-2-yl)pyridine or 3-(azetidin-2-ylmethoxy)pyridine is present in a concentration from about 0.05% w/w to about 4% by weight, such as, e.g., from about 0.05% w/w to about 3.5%, from about 0.07% to about 2.5%, from about 0.1% to about 2.0%, from about 0.1% to about 1.5%, or from about 0.1% to about 1% by weight, calculated as the free base and based on the total weight of the aerosol generating material. One of skill in the art will recognize that the amount of any particular optionally substituted 3-(azetidin-2-yl)pyridine or 3-(azetidin-2-ylmethoxy)pyridine present in the aerosol generating material may vary based on the potency of the compound, the aerosol generating material matrix, and the desired physiological effect for the aerosol generating material.

In some embodiments, the amount of optionally substituted 3-(azetidin-2-yl)pyridine or 3-(azetidin-2-ylmethoxy)pyridine in the aerosol generating material is determined by potency relative to nicotine. For example, in some embodiments, the amount of optionally substituted 3-(azetidin-2-yl)pyridine or 3-(azetidin-2-ylmethoxy)pyridine is based on the potency factor as described above for substituted 3-(1-methylpyrrolidin-2-yl)pyridines. In some embodiments, an optionally substituted 3-(azetidin-2-yl)pyridine or 3-(azetidin-2-ylmethoxy)pyridine of the disclosure has a potency factor from about 0.1 to about 30, such as from about 2 to about 30.

In some embodiments, the amount of optionally substituted 3-(azetidin-2-yl)pyridine or 3-(azetidin-2-ylmethoxy)pyridine in the aerosol generating material is 1 nicotine equivalent. Accordingly, in some embodiments, 1 nicotine equivalent of optionally substituted 3-(azetidin-2-yl)pyridine or 3-(azetidin-2-ylmethoxy)pyridine is an amount by weight from about 10 to about 0.03 times that of nicotine. In some embodiments, 1 nicotine equivalent of optionally substituted 3-(azetidin-2-yl)pyridine or 3-(azetidin-2-ylmethoxy)pyridine is an amount by weight from about 0.5 to about 0.03 times that of nicotine.

For example, a product comprising 2 mg of nicotine, when the nicotine is replaced by an optionally substituted 3-(azetidin-2-yl)pyridine or 3-(azetidin-2-ylmethoxy)pyridine of the disclosure, may include from about 0.06 mg to about 1 mg of the optionally substituted 3-(azetidin-2-yl)pyridine or 3-(azetidin-2-ylmethoxy)pyridine. Similarly, a product comprising 20 mg of nicotine, when the nicotine is replaced by an optionally substituted 3-(azetidin-2-yl)pyridine or 3-(azetidin-2-ylmethoxy)pyridine, may contain from about 0.6 mg to about 100 mg of the optionally substituted 3-(azetidin-2-yl)pyridine or 3-(azetidin-2-ylmethoxy)pyridine.

In some embodiments, the optionally substituted 3-(azetidin-2-yl)pyridine has the structure:

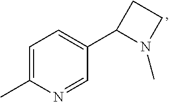

having a potency factor of about 30, meaning that in some embodiments, the amount of optionally substituted 3-(azetidin-2-yl)pyridine present may be about 3% of the amount of nicotine required to achieve the same effect.

In some embodiments, the optionally substituted 3-(azetidin-2-ylmethoxy)pyridine has the structure:

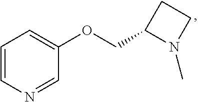

having a potency factor of about 3, meaning that in some embodiments, the amount of 3-(azetidin-2-ylmethoxy)pyridine present may be about 33% of the amount of nicotine required to achieve the same effect.

The optionally substituted 3-(azetidin-2-yl)pyridine or 3-(azetidin-2-ylmethoxy)pyridine may be present in the aerosol generating material as the free base, as a salt with a suitable acid, or in the form of an ion pair with an organic acid. Each of these forms is described further herein below.

### Other Active Agents

In some embodiments, the substituted 3-(1-methylpyrrolidin-2-yl)pyridine, optionally substituted 3-(azetidin-2-yl)pyridine, or optionally substituted 3-(azetidin-2-ylmethoxy)pyridine of the present disclosure is replaced with, or combined with, other active agents that provide the same general pharmacological profile and/or physiological effects of nicotine. Certain of these active agents may be equipotent or even more potent than nicotine with respect to binding affinity to the nicotinic acetylcholine receptor and are expected to preserve the pharmacological effects of nicotine in vivo. Without wishing to be bound by any particular theory, in some embodiments, these compounds are believed to provide the general pharmacological profile and physiological effects of nicotine while offering the potential for one or more of greater potency, reduced product consumption, more rapid and/or complete absorption, and the like.

Example active agents of this type include, without limitation, cytisine, varenicline, acetylcholine, choline, epibatidine, lobeline, analogs thereof, or combinations thereof. Suitable analogs include any of the above-noted compounds having one or more substituents on any of the carbon atoms thereof, with example substituents including alkyl (e.g., C₁-C₃ alkyl), alkoxy, cycloalkyl, alkenyl, alkynyl, aryl, alkylaryl, amino, halogen, and cyano.

In some embodiments, the other active agent is cytisine or an analog thereof. Cytisine is a naturally occurring alkaloid present in certain plant genera, such as Laburnum and Cytisus of the family Fabaceae. Cytisine (CAS Registry No. 485-35-8) has the structure:
[symbol] Cytisine is commercially available and has been utilized in post-Soviet states for more than 40 years as an aid to smoking cessation under the brand name Tabex (Sopharma AD). Cytisine is a partial agonist of the α₄β₂ nicotinic acetylcholine receptor.

In some embodiments, the other active agent is varenicline or an analog thereof. Varenicline is commercially available as Chantix® (Pfizer) and is a medication used as an aid for smoking cessation. Varenicline (CAS Registry No. 249296-44-4) has the structure:
[symbol] Like cytisine, varenicline is a partial agonist of the α₄β₂ nicotinic acetylcholine receptor.

The quantity of the other active agent present in the aerosol generating material may vary. Typically, the other active agent, calculated as the free base, is present in a concentration of at least about 0.001% by weight of the aerosol generating material, such as in a range from about 0.01% to about 10%. In some embodiments, the other active agent is present in a concentration from about 0.05% w/w to about 5% by weight, such as, e.g., from about from about 0.05% w/w. about 0.1% w/w, about 0.2%, about 0.3%, about 0.4%, about 0.5% about 0.6%, about 0.7%, about 0.8%, or about 0.9%, to about 1%, about 2%, about 3%, about 4%, or about 5% by weight, calculated as the free base and based on the total weight of the aerosol generating material. In some embodiments, the other active agent is present in a concentration from about 0.05% w/w to about 4% by weight, such as, e.g., from about 0.05% w/w to about 3.5%, from about 0.07% to about 2.5%, from about 0.1% to about 2.0%, from about 0.1% to about 1.5%, or from about 0.1% to about 1% by weight, calculated as the free base and based on the total weight of the aerosol generating material. One of skill in the art will recognize that the amount of any particular other active agent present in the aerosol generating material may vary based on the potency of the compound, the aerosol generating material matrix, and the desired physiological effect for the aerosol generating material.

The other active agent may be present in the aerosol generating material as the free base, as a salt with a suitable acid, or in the form of an ion pair with an organic acid. Each of these forms is described further herein below.

### Free Base

In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent exhibits sufficient stability, aqueous solubility, and oral bioavailability such that the free base is suitable for inclusion in the aerosol generating material. Accordingly, in some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine or other active agent is present substantially or completely as the free base. In such embodiments, one of skill in the art will recognize that the aerosol generating material is substantially free of acidic components. By “substantially free” it is meant that no acidic component (e.g., inorganic acid, organic acid, or acids capable of salt has been intentionally added, beyond trace amounts that may be present e.g., as an impurity in another component, or small amounts which may be present in certain flavor packages. For example, some embodiments can have less than 0.001% by weight of any acid component, or less than 0.0001%, or even 0% by weight of any acid component, based on the total weight of the aerosol generating material. In some embodiments, the aerosol generating material is completely free of any acid component (i.e., having 0% or having an amount below the limit of detection). In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the free base form and is adsorbed in a carrier such as a microcrystalline cellulose material to form an adsorption complex.

### Salt

In some embodiments, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent can be employed in the form of a salt. A “salt” of such compounds is a form characterized by interaction between the said compound in ionic form and a coformer in ionic form (e.g., an acid) via the transfer of one or more protons from the coformer donor to the compound acceptor. The structure of substituted 3-(1-methylpyrrolidin-2-yl)pyridines, optionally substituted 3-(azetidin-2-yl)pyridines, and optionally substituted 3-(azetidin-2-ylmethoxy)pyridines as disclosed herein are such that they comprise two nitrogen atoms that are capable of accepting protons from a coformer and, accordingly, can be present in non-protonated, mono-protonated, and/or di-protonated form in a given sample. Salts of substituted 3-(1-methylpyrrolidin-2-yl)pyridine, optionally substituted 3-(azetidin-2-yl)pyridine, optionally substituted 3-(azetidin-2-ylmethoxy)pyridine, or other active agent can be provided using the types of ingredients and techniques set forth for nicotine in U.S. Pat. No. 2,033,909 to Cox et al. and Perfetti, Beitrage Tabakforschunglnt., 12: 43-54 (1983), which are incorporated herein by reference Suitable salts are generally water soluble. Suitable acids for formation of salts (mono- and di-) include, but are not limited to, acetic acid, adipic acid, ascorbic acid, capric acid, citric acid, D-glucuronic acid, D-gluconic acid, lactic acid, galactaric acid, hippuric acid, hydrochloric acid, L-aspartic acid, L-glutamic acid, L-glutaric acid, glycerophosphoric acid, glycolic acid, lauric acid, DL-malic acid, L-malic acid; tartaric acid, palmitic acid, phosphoric acid, sebacic acid, stearic acid, succinic acid, sulfuric acid, thiocyanic acid, (+)-camphoric acid, 1,5-naphthalenedisulfonic acid, 1-hydroxy-2-naphthoic, 2,5-dihydroxybenzoic acid, benzenesulfonic acid, benzoic acid, caprylic acid, cyclamic acid, ethanesulfonic acid, fumaric acid, D-glucoheptonic acid, 4-hydroxybenzoic acid, isobutyric acid, ketoglutaric acid, 2-ketobutyric acid, lactobionic acid, maleic acid, malonic acid, methanesulfonic acid, naphthalene-2-sulfonic acid, nicotinic acid, oleic acid, orotic acid, oxalic acid, pamoic acid, pivalic acid, propionic acid, L-pyroglutamic acid, p-toluenesulfonic acid, (1S)-camphor-10-sulfonic acid, 4-acetamidobenzoic acid, 4-aminosalicylic acid, N-acetyl-4-aminosalicylic acid, caproic acid, dichloroacetic acid, hydrobromic acid, DL-mandelic acid, L-mandelic acid, nitric acid, formic acid, salicylic acid, cinnamic acid, undecylenic acid, isothionic acid, lauric acid, 2-hydroxybenzoic acid, trans-2-hexanoic acid, trimesic acid, 5-nitroisophthalic acid and zinc chloride monohydrate (forming a hydrated zinc chloride complex salt). In some embodiments, the salt is with an organic acid as described herein below.

In some embodiments, a hydrophilic acid is chosen so as to increase water solubility and/or decrease lipophilicity of the salt. Lipophilicity of a salt of a compound as disclosed herein can also be expressed as log D, which is the logarithm of the distribution coefficient, a measure of the pH-dependent differential solubility between an octanol phase and an aqueous phase of all species (ionized and un-ionized) in an octanol/aqueous system, represented by the formula:

$$log {D}_{oct / wat} = log ( \frac{{[ solute ]}_{octanol}}{{[ solute ]}_{water}^{ionized} + {[ solute ]}_{water}^{neutral}} ) .$$

Log D is a commonly used descriptor for the lipophilicity of ionizable compounds. Log D values can be calculated using commercial software or may be determined experimentally in a similar manner to log P but instead of using water, the aqueous phase is adjusted to a specific pH using a buffer. Log D is pH dependent and therefore requires that the pH at which the log D was measured be specified.

When the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the form of a salt, it is generally preferred that the salt have a relatively low log D, indicative of good water solubility. Without wishing to be bound by theory, it is believed that highly water-soluble salt forms may exhibit a high rate of dissolution, which may be favorable in certain embodiments. Accordingly, in some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent salt has a log D from about −1.0 to about 3 at a pH in a range from about 3 to about 11, such as from about −0.5 to about 2, about −0.3 to about 1, or about −0.1 to about 0.

In some embodiments, the selection of acid used to make a salt is performed on the basis of sensory effects of the salt, such as taste. Surprisingly, according to the present disclosure, it has been found that salts of certain organic acids, such as galactaric acid, offer a better taste sensation relative to salts of acids such as tartaric or phthalic acids.

In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the form of a salt with tartaric acid, succinic acid, orotic acid, fumaric acid, pyroglutamic acid, or galactaric acid. In some embodiments, the substituted 3-(1-methylpyrrolidin-2-yl)pyridine, optionally substituted 3-(azetidin-2-yl)pyridine, or optionally substituted 3-(azetidin-2-ylmethoxy)pyridine is present in the form of a salt with succinic acid or galactaric acid.

The stoichiometry of the salts as described herein can vary. For example, in some embodiments, the stoichiometry can range from about 5:1 to about 1:5 molar equivalents of compound:acid, including any whole or fractional value in between. In some embodiments, the molar ratio of compound to acid is about 2:1, about 1:1, or about 1:2. In some embodiments, the compound is present in the form of a bitartrate salt. Hydrates and other solvates of salts are further contemplated herein.

The salts as described herein can, in some embodiments, exist in various polymorphic and pseudopolymorphic forms. Polymorphism is the ability of a crystalline material to exist in more than one form or crystal structure. Polymorphism can result, e.g., from the existence of different crystal packing structures (packing pleomorphism) or from the existence of different conformers of the same molecule (conformational polymorphism). Pseudopolymorphism is the result of hydration or solvation of a material and is also referred to as solvomorphism.

### Organic Acid

In some embodiments, the aerosol generating material comprises an organic acid. In some embodiments, the organic acid is a carboxylic acid or a sulfonic acid. The carboxylic acid or sulfonic acid functional group may be attached to any alkyl, cycloalkyl, heterocycloalkyl, aryl, or heteroaryl group having, for example, from one to twenty carbon atoms (C₁-C₂₀). In some embodiments, the organic acid is an alkyl, cycloalkyl, heterocycloalkyl, aryl, or heteroaryl carboxylic or sulfonic acid.

As used herein, “alkyl” refers to any straight chain or branched chain hydrocarbon. The alkyl group may be saturated (i.e., having all sp³ carbon atoms), or may be unsaturated (i.e., having at least one site of unsaturation). As used herein, the term “unsaturated” refers to the presence of a carbon-carbon, sp² double bond in one or more positions within the alkyl group. Unsaturated alkyl groups may be mono- or polyunsaturated. Representative straight chain alkyl groups include, but are not limited to, methyl, ethyl, n-propyl, n-butyl, n-pentyl, and n-hexyl. Branched chain alkyl groups include, but are not limited to, isopropyl, sec-butyl, isobutyl, tert-butyl, isopentyl, and 2-methylbutyl. Representative unsaturated alkyl groups include, but are not limited to, ethylene or vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. An alkyl group can be unsubstituted or substituted.

“Cycloalkyl” as used herein refers to a carbocyclic group, which may be mono- or bicyclic. Cycloalkyl groups include rings having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted, and may include one or more sites of unsaturation (e.g., cyclopentenyl or cyclohexenyl).

The term “aryl” as used herein refers to a carbocyclic aromatic group. Examples of aryl groups include, but are not limited to, phenyl and naphthyl. An aryl group can be unsubstituted or substituted.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl group comprises up to 20 carbon atoms and from 1 to 3 heteroatoms selected from N, O, and S. A heteroaryl or heterocycloalkyl may be a monocycle having 3 to 7 ring members (for example, 2 to 6 carbon atoms and 1 to 3 heteroatoms selected from N, O, and S) or a bicycle having 7 to 10 ring members (for example, 4 to 9 carbon atoms and 1 to 3 heteroatoms selected from N, O, and S), for example: a bicyclo[4,5], [5,5], [5,6], or [6,6] system. Examples of heteroaryl groups include by way of example and not limitation, pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl. Examples of heterocycloalkyls include by way of example and not limitation, dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl. Heteroaryl and heterocycloalkyl groups can be unsubstituted or substituted.

“Substituted” as used herein and as applied to any of the above alkyl, aryl, cycloalkyl, heteroaryl, heterocyclyl, means that one or more hydrogen atoms are each independently replaced with a substituent. Typical substituents include, but are not limited to, —Cl, Br, F, alkyl, —OH, —OCH₃, NH₂, —NHCH₃, —N(CH₃)₂, —CN, —NC(═O)CH₃, —C(═O)—, —C(═O)NH₂, and —C(═O)N(CH₃)₂. Wherever a group is described as “optionally substituted,” that group can be substituted with one or more of the above substituents, independently selected for each occasion. In some embodiments, the substituent may be one or more methyl groups or one or more hydroxyl groups.

In some embodiments, the organic acid is an alkyl carboxylic acid. Non-limiting examples of alkyl carboxylic acids include formic acid, acetic acid, propionic acid, butyric acid, valeric acid, caproic acid, heptanoic acid, octanoic acid, nonanoic acid, decanoic acid, undecanoic acid, dodecanoic acid, stearic acid, oleic acid, linoleic acid, linolenic acid, and the like.

In some embodiments, the organic acid is an alkyl sulfonic acid. Non-limiting examples of alkyl sulfonic acids include propanesulfonic acid, heptanesulfonic acid, and octanesulfonic acid.

In some embodiments, the alkyl carboxylic or sulfonic acid is substituted with one or more hydroxyl groups. Non-limiting examples include glycolic acid, 4-hydroxybutyric acid, and lactic acid.

In some embodiments, an organic acid may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid may include more than one carboxylic acid group and one or more hydroxyl groups. Non-limiting examples of such acids include tartaric acid, citric acid, and the like.

In some embodiments, the organic acid is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid.

Further non-limiting examples of organic acids which may be useful in certain embodiments include 2-(4-isobutylphenyl)propanoic acid, 2,2-dichloroacetic acid, 2-hydroxyethanesulfonic acid, 2-oxoglutaric acid, 4-acetamidobenzoic acid, 4-aminosalicylic acid, adipic acid, ascorbic acid (L), aspartic acid (L), alpha-methylbutyric acid, camphoric acid (+), camphor-10-sulfonic acid (+), cinnamic acid, cyclamic acid, dodecylsulfuric acid, ethane-1,2-disulfonic acid, ethanesulfonic acid, furoic acid, galactaric acid, gentisic acid, glucoheptonic acid, gluconic acid, glucuronic acid, glutamic acid, glycerophosphoric acid, glycolic acid, hippuric acid, isobutyric acid, isovaleric acid, lactobionic acid, lauric acid, levulinic acid, malic acid, malonic acid, mandelic acid, methanesulfonic acid, naphthalene-1,5-disulfonic acid, naphthalene-2-sulfonic acid, oleic acid, palmitic acid, pamoic acid, phenylacetic acid, pyroglutamic acid, pyruvic acid, sebacic acid, stearic acid, and undecylenic acid. Examples of suitable acids include, but are not limited to, the list of organic acids in Table 1.

TABLE 1
Non-limiting examples of suitable organic acids
Acid Name

benzoic acid
phenylacetic
p-toluic acid
ethyl benzoic acid
isopropyl benzoic acid
4-phenylbutyric
2-(4-Isobutylphenyl)propanoic acid
2-napthoxyacetic acid
napthylacetic acid
heptanoic acid
octanoic acid
nonanoic acid
decanoic acid
9-deceneoic acid
2-deceneoic acid
10-undecenoic acid
dodecandioic acid
dodecanoic acid
myristic acid
palmitic acid
stearic acid
cyclohexanebutanoic acid
1-heptanesulfonic acid
1-octanesulfonic acid
1-nonanesulfonic acid

The selection of organic acid may depend on certain properties. For example, an organic acid should be one recognized as safe for human consumption, and which has acceptable flavor, odor, volatility, stability, and the like. Determination of appropriate organic acids is within the purview of one of skill in the art. In some embodiments, the acid is benzoic acid. In some embodiments, the acid is levulinic acid. In some embodiments, the acid is lactic acid. In some embodiments, the acid is one or more of benzoic acid, lactic acid, and levulinic acid. In some embodiments, benzoic acid and levulinic acid are used, such as in a weight ratio of benzoic acid to levulinic acid of 10:1 to 1:1 or 8:1 to 2:1 or 6:1 to 3:1.

In some embodiments, more than one organic acid may be present. For example, the aerosol generating material may comprise two, or three, or four, or more organic acids. Accordingly, reference herein to “an organic acid” contemplates mixtures of two or more organic acids. The relative amounts of the multiple organic acids may vary. For example, an aerosol modifying agent may comprise equal amounts of two, or three, or more organic acids, or may comprise different relative amounts. Without wishing to be bound by theory, it is believed that a combination of different organic acids may provide a concentration of any single organic acid in the aerosol generating material which remains below the threshold which would be found objectionable from a sensory perspective.

The amount of organic acid present in the aerosol generating material, relative to the substituted 3-(1-methylpyrrolidin-2-yl)pyridine, 3-(azetidin-2-yl)pyridine, or 3-(azetidin-2-ylmethoxy)pyridine (e.g., 2-methyl-5-(1-methylpyrrolidin-2-yl)pyridine), may vary. In some embodiments, the aerosol generating material comprises from about 0.05, about 0.1, about 1, about 1.5, about 2, or about 5, to about 10, about 15, or about 20 molar equivalents of the organic acid relative to the substituted 3-(1-methylpyrrolidin-2-yl)pyridine, 3-(azetidin-2-yl)pyridine, or 3-(azetidin-2-ylmethoxy)pyridine, calculated as the free base of the 3-(1-methylpyrrolidin-2-yl)pyridine, 3-(azetidin-2-yl)pyridine, or 3-(azetidin-2-ylmethoxy)pyridine.

In some embodiments, the aerosol generating material comprises from about 2 to about 10, or from about 2 to about 5 molar equivalents of the organic acid relative to the substituted 3-(1-methylpyrrolidin-2-yl)pyridine, 3-(azetidin-2-yl)pyridine, or 3-(azetidin-2-ylmethoxy)pyridine, on a free-base basis. In some embodiments, the organic acid is present in a molar ratio with the substituted 3-(1-methylpyrrolidin-2-yl)pyridine, 3-(azetidin-2-yl)pyridine, or 3-(azetidin-2-ylmethoxy)pyridine from about 2, about 3, about 4, or about 5, to about 6, about 7, about 8, about 9, or about 10. In embodiments wherein more than one organic acid it is to be understood that such molar ratios reflect the totality of the organic acids present.

In some embodiments, the aerosol generating material comprises one or more of the organic acids described herein above. The substituted 3-(1-methylpyrrolidin-2-yl)pyridine, 3-(azetidin-2-yl)pyridine, or 3-(azetidin-2-ylmethoxy)pyridine and the one or more organic acids may be present independently, in the form of a salt, or as a cocrystal. In some embodiments, the aerosol generating material comprises one or more of lactic acid, levulinic acid, benzoic acid, succinic acid, galactaric acid, orotic acid, fumaric acid, pyroglutamic acid, and tartaric acid, and at least a portion of said acid is present in the form of a salt with the substituted 3-(1-methylpyrrolidin-2-yl)pyridine, 3-(azetidin-2-yl)pyridine, or 3-(azetidin-2-ylmethoxy)pyridine.

In some embodiments, the aerosol generating material comprises a lactic acid salt of the substituted 3-(1-methylpyrrolidin-2-yl)pyridine.

In some embodiments, the aerosol generating material comprises a levulinic acid salt of the substituted 3-(1-methylpyrrolidin-2-yl)pyridine.

In some embodiments, the aerosol generating material comprises a galactaric acid salt of the substituted 3-(1-methylpyrrolidin-2-yl)pyridine.

In some embodiments, the aerosol generating material comprises a tartaric acid salt of the substituted 3-(1-methylpyrrolidin-2-yl)pyridine.

The stoichiometry of such lactate, levulinate, tartrate, and galactarate salts may vary. Accordingly, the molar ratio of lactic, levulinic, tartaric, or galactaric acid to substituted 3-(1-methylpyrrolidin-2-yl)pyridine may be in a range from about 3:1 to about 1:3, including any whole or fractional value in between, such as for example about 2:1, about 1.5:1, about 1:1, about 1:1.5, or about 1:2.

In some embodiments, the aerosol generating material may be substantially or completely free of organic acids (i.e., having less than 0.001% by weight of organic acid, or less than 0.0001%, or even 0% by weight of organic acid, based on the total weight of the aerosol generating material, or as having an amount of organic acid below the limit of detection).

### Resin Complex

In some embodiments, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent may be present in the form of a polymer complex, where the compound is bound to an acidic polymer. The polymer of such a complex can be any polymer (including homopolymers or all types of copolymers) with acidic functionalities, e.g., a polymeric cation exchange resin. In some embodiments, the polymer comprises acidic sites that can be classified as strongly acidic, weakly acidic, or of intermediate acidity (depending, e.g., on the strength of the acid from which they are derived). In some embodiments, the polymer comprises weakly acidic sites and can be referred to as a weakly acidic cation exchange resin. Non-limiting examples of acidic sites include, e.g., carboxylic acids, sulfonic acids, phosphonous acids, phosphonic acids, phosphoric acids, iminodiacetic acids, and phenolic groups (e.g., as disclosed in Adams et al., J. Soc. Chem. Ind. 54, IT (1935), which is incorporated herein by reference). Suitable polymers include, but are not limited to, addition polymers of styrene and divinylbenzene, divinylbenzene and methacrylic acid, divinylbenzene and acrylic acid, phenolic resins, or cellulose, dextran or pectin cross-linked with, e.g., epichlorohydrin. In some embodiments, the polymer comprises cross-linked moieties. Various acidic ion-exchange resins which are known in the art and are suitable for formation of complexes, include, but are not limited to, polymethacrylic acid resins such as DuPont™ Amberlite™ IRP64, DuPont™ Amberlite™ IRP69, Purolite™ C115HMR, Doshion™ P551, and polyacrylic carbomers, such as Carbopol 974P. See, for example, U.S. Pat. No. 3,901,248 to Lichtneckert et al., which is incorporated herein by reference. In some embodiments, when the substituted 3-(1-methylpyrrolidin-2-yl)pyridine, optionally substituted 3-(azetidin-2-yl)pyridine, or optionally substituted 3-(azetidin-2-ylmethoxy)pyridine is present in the form of a polymer complex, the aerosol generating material further comprises a divalent metal buffer, such as a calcium or magnesium salt (e.g., carbonate, bicarbonate, oxide, acetate, or the like).

### Cocrystal

In some embodiments, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent may be present in the form of a co-crystal with at least one other component (“coformer”), both in neutral form. Specifically, as defined in a US FDA industry guidance document, a co-crystal is a solid that is a crystalline material composed of two or more molecules in the same crystal lattice, where the components are in a neutral state and interact via nonionic interactions. See U.S. Department of Health and Human Services, Food and Drug Administration, Guidance for Industry: Regulatory Classification of Pharmaceutical Co-Crystals (April 2013), which is incorporated herein by reference. This form is different and distinct from both salts and ion pairs, each described herein. Specifically, co-crystals can generally be distinguished from salts (and ion pairs) by the absence of a proton transfer between the components (i.e., a substituted 3-(1-methylpyrrolidin-2-yl)pyridine and the one or more coformers) in a co-crystal. The crystalline structure of the co-crystal is generally held together by freely reversible, non-covalent interactions. Co-crystals typically comprise the substituted 3-(1-methylpyrrolidin-2-yl)pyridine, optionally substituted 3-(azetidin-2-yl)pyridine, or optionally substituted 3-(azetidin-2-ylmethoxy)pyridine and coformer in a defined stoichiometric ratio. In some embodiments, co-crystals can encompass hydrates, solvates, and clathrates. Co-crystals can comprise the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent in combination with an organic and/or an inorganic coformer.

Examples of suitable coformers include, but are not limited to, acetamidobenzoic acid, L-proline, tromethamine, urea, xylitol, caffeine, glycine/glycine anhydride, vanillin, methyl 4-hydroxybenzoate(methylparaben), succinimide, L-alanine, mannitol, L-phenylalanine, saccharin, propylparaben, N-methylglucamine, L-tyrosine, gentisic acid, sorbic acid, benzoic acid, L-methionine, maltol, L-lysine, tromethamine, nicotinamide, isonicotinamide, phenylalanine, benzoquinone, terephthalaldehyde, 4-hydroxybenzoic acid, pyruvic acid, 1-hydroxy-2-naphthoic acid, 4-aminobenzoic acid, vanillic acid, ethyl vanillin, isonicotinic acid, gallic acid, menthol (e.g., racemic menthol or (−)-menthol), paracetamol, aspirin, ibuprofen, naproxen, ketoprofen, flurbiprofen, glucose, serine, malic acid, acetamide, sulfacetamide, benzoic acid, creatine, 2-hydroxyethanesulfonic acid, clofibric acid, taurine (tauric acid), iproniazid, L-histadine, L-arginine, L-asparagine, glutamine, L-cysteine, alanine, valine, isoleucine, leucine, morpholine, theronine, N-methylglucamine, 3-hydroxy-2-oxopropionic acid; 2-oxobutyric acid (2-ketobutyric acid), 3-methyl-2-oxobutanoic acid; 3-methyl-2-oxopentanoic acid; 4-methyl-2-oxopentanoic acid; and 2-oxopentanedioic acid, 2-oxo-3-phenylpropionic acid; 5-oxooctanoic acid; and 5-oxodecanoic acid, aldonic acids (e.g., glyceric acid, xylonic acid, gluconic acid, and ascorbic acid), ulosonic acids (e.g., neuraminic acid and ketodeoxyoctulosonic acid), uronic acids (e.g., glucuronic acid, galacturonic acid, and iduronic acid), aldaric acids (e.g., tartaric acid, meso-galactaric acid/mucic acid, and D-glucaric acid/saccharic acid), galactaric acid), and polyfunctional aromatic acids.

In some embodiments, the conformer is a polyfunctional aromatic acid. Polyfunctional aromatic acids often comprise a substituted or unsubstituted phenyl group as the aromatic component, but can alternatively comprise another aromatic moiety, e.g., pyridine, pyrazine, imidazole, pyrazole, oxazole, thiophene, naphthalene, anthracene, and phenanthrene. Substituents on the optionally substituted aromatic acids may be any type of substituent, including, but not limited to, halo (e.g., Cl, F, Br, and I); alkyl, halogenated alkyl (e.g., CF₃, 2-Br-ethyl, CH₂F, CH₂Cl, CH₂CF₃, or CF₂CF₃); alkenyl, hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate groups. Example polyfunctional aromatic acids can be, for example:
substituted and unsubstituted aromatic dicarboxylic acids (e.g., 1,2-benzenedicarboxylic acid (phthalic acid), 1,3-benzenedicarboxylic acid (isophthalic acid), 1,4-benzenedicarboxylic acid (terephthalic acid), 2-iodo-1,3-benzenedicarboxylic acid, 2-hydroxy-1,4-benzenedicarboxylic acid, 2-nitro-1,4-benzenedicarboxylic acid, 3-fluoro-1,2-benzenedicarboxylic acid, 3-amino-1,2-benzenedicarboxylic acid, 3-nitro-1,2-benzenedicarboxylic acid, 4-bromo-1,3-benzenedicarboxylic acid, 4-hydroxy-1,3-benzenedicarboxylic acid, 4-amino-1,2-benzenedicarboxylic acid, 4-nitro-1,2-benzenedicarboxylic acid, 4-sulfo-1,2-benzenedicarboxylic acid, 4-amino-1,3-benzenedicarboxylic acid, 5-bromo-1,3-benzenedicarboxylic acid, 5-hydroxy-1,3-benzenedicarboxylic acid, 5-amino-1,3-benzenedicarboxylic acid, 5-nitro-1,3-benzenedicarboxylic acid, 5-ethynyl-1,3-benzenedicarboxylic acid, 5-cyano-1,3-benzenedicarboxylic acid, 5-nitro-1,3-benzenedicarboxylic acid, 2,5-hydroxy-1,4-benzenedicarboxylic acid, and 2,3,5,6-tetrafluoro-1,4-benzenedicarboxylic acid;
substituted and unsubstituted hydroxybenzoic acids (e.g., 2-hydroxybenzoic acid (salicylic acid), 3-hydroxybenzoic acid, 4-hydroxybenzoic acid, 2-methyl-4-hydroxybenzoic acid, 3-tert-butyl-4-hydroxybenzoic acid, 4-ethoxy-2-hydroxybenzoic acid, 3-chloro-5-hydroxybenzoic acid, 5-chloro-2-hydroxybenzoic acid, 3-bromo-4-hydroxybenzoic acid, 3-bromo-5-hydroxybenzoic acid, 4-bromo-2-hydroxybenzoic acid, 5-bromo-2-hydroxybenzoic acid, 2-fluoro-5-hydroxybenzoic acid, 3-fluoro-4-hydroxybenzoic acid, 3-fluoro-2-hydroxybenzoic acid, 3-fluoro-5-hydroxybenzoic acid, 2-fluoro-6-hydroxybenzoic acid, 4-fluoro-3-hydroxybenzoic acid, 2-fluoro-4-hydroxybenzoic acid, 5-fluoro-2-hydroxybenzoic acid, 2-amino-3-hydroxybenzoic acid, 2-amino-5-hydroxybenzoic acid, 3-amino-2-hydroxybenzoic acid, 3-amino-4-hydroxybenzoic acid, 3-amino-5-hydroxybenzoic acid, 4-amino-2-hydroxybenzoic acid, 4-amino-3-hydroxybenzoic acid, 5-amino-2-hydroxybenzoic acid (mesalamine), 5-aminomethyl-2-hydroxybenzoic acid, 4-formyl-3-hydroxybenzoic acid, 3-formyl-4-hydroxybenzoic acid, 5-(acetylamino)-2-hydroxybenzoic acid), 4-nitro-2-hydroxybenzoic acid, 3,5-diethyl-4-hydroxybenzoic acid, 3,5-di-tert-butyl-4-hydroxybenzoic acid, 3,5-diisopropyl-2-hydroxybenzoic acid, 3,4-dimethoxy-4-hydroxybenzoic acid (syringic acid), 3,5-dichloro-2-hydroxybenzoic acid, 3,5-dichloro-4-hydroxybenzoic acid, 3,6-dichloro-2-hydroxybenzoic acid, 2,3-difluoro-4-hydroxybenzoic acid, 3,4-difluoro-2-hydroxybenzoic acid, 3,5-dibromo-2-hydroxybenzoic acid, 3,5-diodo-2-hydroxybenzoic acid, 4-amino-5-chloro-2-hydroxybenzoic acid, 3,5-dinitro-2-hydroxybenzoic acid, 2,4,6-tribromo-2-hydroxybenzoic acid, 2,3,5,6-tetrafluoro-4-hydroxybenzoic acid, and 2,3,4,5-tetrafluoro-6-hydroxybenzoic acid);
substituted and unsubstituted dihydroxybenzoic acids (e.g., 2,3-dihydroxybenzoic acid (pyrocatechuic acid/hypogallic acid), 2,4-dihydroxybenzoic acid (β-resorcylic acid), 2,5-dihydroxybenzoic acid (gentisic acid/hydroquinonecarboxylic acid), 2,6-dihydroxybenzoic acid (γ-resorcylic acid), 3,4-dihydroxybenzoic acid (protocatechuic acid), 3,5-dihydroxybenzoic acid (α-resorcylic acid), 4-hydroxy-3-methoxybenzoic acid (vanillic acid), 6-methyl-2,4-dihydroxybenzoic acid (orsellenic acid), 4-bromo-3,5-dihydroxybenzoic acid, 5-bromo-2,4-dihydroxybenzoic acid, 5-bromo-3,4-dihydroxybenzoic acid, 6-carboxymethyl-2,3-dihydroxybenzoic acid, 3,5-dibromo-2,4-dihydroxybenzoic acid, 3,5-dichloro-2,6-dihydroxybenzoic acid, and 5-amino-3-chloro-2,4-dihydroxybenzoic acid);
substituted and unsubstituted trihydroxybenzoic acids (e.g., 2,3,4-trihydroxybenzoic acid, 2,4,5-trihydroxybenzoic acid, 2,4,6-trihydroxybenzoic acid (phloroglucinol carboxylic acid), and 3,4,5-trihydroxybenzoic acid (gallic acid));
substituted and unsubstituted aromatic tricarboxylic acids (e.g., 1,2,3-benzenetricarboxylic acid, 1,2,4-benzenetricarboxylic acid (trimellitic acid); and
substituted and unsubstituted aromatic tetracarboxylic acids (e.g., 1,2,3,4-benzenetetracarboxylic acid (mellophanic acid) and 1,2,4,5-benzenetetracarboxylic acid (pyromellitic acid). Further contemplated are various combinations of any of the foregoing acids.

In some embodiments, the coformer is L-malic acid, succinic acid, or a combination thereof. In some embodiments, the coformer is 1,1,6,6-tetraphenyl-2,4-hexidiyne-1,6-diol. In some embodiments, the coformer is di-iodotetrafluoro benzene, 4,4′-diiodooctafluorobiphenyl, or 1,4-bis(diphenylhydroxymethyl)benzene. In some embodiments, the coformer is orotic acid.

In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the form of a salt co-crystal. A “salt co-crystal” is a type of hybrid structure with both salt and co-crystal characteristics. Typically, a substituted 3-(1-methylpyrrolidin-2-yl)pyridine, optionally substituted 3-(azetidin-2-yl)pyridine, optionally substituted 3-(azetidin-2-ylmethoxy)pyridine molecule, or other active agent within a salt co-crystal is associated with at least two coformers (which may be the same or different), wherein one coformer is in ionic form (e.g., an acid) and transfers a proton to the substituted 3-(1-methylpyrrolidin-2-yl)pyridine, optionally substituted 3-(azetidin-2-yl)pyridine, optionally substituted 3-(azetidin-2-ylmethoxy)pyridine molecule, or other active agent, and wherein a second coformer does not transfer a proton to the substituted 3-(1-methylpyrrolidin-2-yl)pyridine, optionally substituted 3-(azetidin-2-yl)pyridine, optionally substituted 3-(azetidin-2-ylmethoxy)pyridine molecule, or other active agent. Suitable acids and coformers are generally those described herein above with respect to salts and co-crystals.

The stoichiometry of the co-crystals and salt co-crystals described herein can vary. For example, in certain embodiments, where two components are present, the stoichiometry can range in certain embodiments from about 5:1 to about 1:5 compound:coformer. Where more than one coformer is used to form a co-crystal or salt co-crystal, the ratios of the coformers with respect to both the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent and to one another can also vary.

The co-crystals and salt co-crystals described herein can, in some embodiments, exist in various polymorphic and pseudopolymorphic forms, as well as solvates and hydrates.

In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the form of a salt-co-crystal. In some embodiments, the salt-co-crystal is a bis-orotic acid salt-co-crystal. In some embodiments, the bis-orotic acid salt-co-crystal is a hemi-hydrate.

### Ion Pairing

In some embodiments, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the form of an ion pair. Ion pairing describes the partial association of oppositely charged ions in relatively concentrated solutions to form distinct chemical species called ion pairs. The strength of the association (i.e., the ion pairing) depends on the electrostatic force of attraction between the positive and negative ions (e.g., a substituted 3-(1-methylpyrrolidin-2-yl)pyridine and the conjugate base of a suitable acid). By “conjugate base” is meant the base resulting from deprotonation of the corresponding acid (e.g., benzoate is the conjugate base of benzoic acid). In embodiments comprising ion pairing, on average, a certain population of these ion pairs exists at any given time, although the formation and dissociation of ion pairs is continuous. In some embodiments, in the aerosol generating material as disclosed herein, and/or upon oral use of said aerosol generating material (e.g., upon contact with saliva), the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent and the conjugate base of an acid exist at least partially in the form of an ion pair. Ion pairing is further described in, for example, International Patent Application Publication No. WO2021/050741 to Poole et al., and US Application Publication Nos. 2021/0068447 to Keller et al., 2023/0138306A1 to Zawadzki et al., and 2022/0346434 to Von Cosmos et al., each of which is incorporated herein by reference.

One of skill in the art will recognize that the extent of ion pairing in the disclosed aerosol generating material, both before and during use by the consumer, may vary based on, for example, pH, the nature of the acid, the concentration of substituted 3-(1-methylpyrrolidin-2-yl)pyridine, optionally substituted 3-(azetidin-2-yl)pyridine, optionally substituted 3-(azetidin-2-ylmethoxy)pyridine, or other active agent, the concentration of the acid or conjugate base of the acid present in the aerosol generating material, the moisture content of the aerosol generating material, the ionic strength of the aerosol generating material, and the like. One of skill in the art will also recognize that ion pairing is an equilibrium process influenced by the foregoing variables. Accordingly, quantification of the extent of ion pairing is difficult or impossible by calculation or direct observation. However, the presence of ion pairing may be demonstrated through surrogate measures, such as partitioning of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent between octanol and water, or by performing membrane permeation studies of aqueous solutions of, for example, the substituted 3-(1-methylpyrrolidin-2-yl)pyridine plus acids and/or their conjugate bases. An octanol-water partitioning favoring distribution of an ion pair into octanol is predictive of good absorption of the substituted 3-(1-methylpyrrolidin-2-yl)pyridine, optionally substituted 3-(azetidin-2-yl)pyridine, optionally substituted 3-(azetidin-2-ylmethoxy)pyridine (e.g., 2-methyl-5-(1-methylpyrrolidin-2-yl)pyridine), or other active agent through the oral mucosa. However, as described above, in some embodiments, the properties of the substituted 3-(1-methylpyrrolidin-2-yl)pyridine, optionally substituted 3-(azetidin-2-yl)pyridine, optionally substituted 3-(azetidin-2-ylmethoxy)pyridine (e.g., 2-methyl-5-(1-methylpyrrolidin-2-yl)pyridine), or other active agent are such that no ion pairing is required, and accordingly, the aerosol generating material is substantially or completely free of any ion pairing. By “substantially free” it is meant that no measurable degree of ion pairing is present.

In embodiments where ion pairing is desired, the aerosol generating material comprises an organic acid, an alkali metal salt of an organic acid, or both. In such embodiments, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is associated with at least a portion of the organic acid, the alkali metal salt thereof, or a combination thereof in the form an ion pair. As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific composition agent as opposed to merely being inherently present as a component of another composition ingredient (e.g., the small amount of organic acid which may inherently be present in a composition ingredient). For the avoidance of doubt, reference herein to an “organic acid” is intended to distinguish the acid present in ion paired forms over the acid which may be present in salts, co-crystal, and salt co-crystals. While one of skill in the art will recognize that certain organic acids suitable for formation of ion pairs overlap with those identified as suitable for salt or co-crystal formation, it is to be understood that the particular acid used for each of salts, co-crystals, and ion pairs are to be selected specifically for each such embodiment, and reference herein to an organic acid is specific to acids suitable for ion pairing. Accordingly, the presence in the aerosol generating material of an organic acid as defined below is to be interpreted solely with respect to ion pairing, even if such organic acid is also suitable for salt formation or co-crystal formation, and the presence of such an organic acid does not imply that a salt or co-crystal is present unless explicitly identified. Further, in embodiments where there is no ion pairing intended, the aerosol generating material may be characterized as substantially or completely free of organic acids (i.e., having less than 0.001% by weight of organic acid, or less than 0.0001%, or even 0% by weight of organic acid, based on the total weight of the aerosol generating material, or as having an amount of organic acid below the limit of detection). This is not to be interpreted as meaning that the aerosol generating material is substantially or completely free of substituted 3-(1-methylpyrrolidin-2-yl)pyridine salts or substituted 3-(1-methylpyrrolidin-2-yl)pyridine co-crystals unless explicitly recited.

The amount of organic acid or alkali metal salt thereof present in the aerosol generating material, relative to the substituted 3-(1-methylpyrrolidin-2-yl)pyridine, optionally substituted 3-(azetidin-2-yl)pyridine, optionally substituted 3-(azetidin-2-ylmethoxy)pyridine (e.g., 2-methyl-5-(1-methylpyrrolidin-2-yl)pyridine), or other active agent, may vary. Generally, as the concentration of the organic acid (or the conjugate base thereof) increases, the percent of substituted 3-(1-methylpyrrolidin-2-yl)pyridine, optionally substituted 3-(azetidin-2-yl)pyridine, optionally substituted 3-(azetidin-2-ylmethoxy)pyridine, or other active agent that is ion paired with the organic acid increases. This typically increases the partitioning of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent in the form of an ion pair, into octanol versus water as measured by the log P (the log₁₀ of the partitioning coefficient). In some embodiments, the aerosol generating material comprises from about 0.05, about 0.1, about 1, about 1.5, about 2, or about 5, to about 10, about 15, or about 20 molar equivalents of the organic acid, the alkali metal salt thereof, or the combination thereof, relative to the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent, calculated as the free base of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent.

In some embodiments, the aerosol generating material comprises from about 2 to about 10, or from about 2 to about 5 molar equivalents of the organic acid, the alkali metal salt thereof, or the combination thereof, relative to the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent on a free-base basis. In some embodiments, the organic acid, the alkali metal salt thereof, or the combination thereof, is present in a molar ratio with the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent from about 2, about 3, about 4, or about 5, to about 6, about 7, about 8, about 9, or about 10. In embodiments wherein more than one organic acid, alkali metal salt thereof, or both, are present, it is to be understood that such molar ratios reflect the totality of the organic acids present. In some embodiments, the aerosol generating material comprises benzoic acid and sodium benzoate wherein a total amount of benzoate (i.e., benzoic acid and benzoate) is in a molar ratio in a range from about 3 to about 5 relative to the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent. In some embodiments, the molar ratio of the total amount of benzoate to the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is about 3.2 or about 4.8.

In some embodiments, the organic acid inclusion is sufficient to provide a pH of from about 4.0 to about 9.0, such as from about 4.5 to about 7.0, or from about 5.5 to about 7.0, from about 4.0 to about 5.5, or from about 7.0 to about 9.0. Reference herein to “a pH” means the pH of an aqueous solution of the aerosol generating material prepared by dissolving or suspending 5 grams of aerosol generating material in 95 grams of water and measuring the pH of the resulting solution with a calibrated pH meter.

In some embodiments, the organic acid inclusion is sufficient to provide a pH of from about 4.5 to about 6.5, for example, from about 4.5, about 5.0, or about 5.5, to about 6.0, or about 6.5. In some embodiments, the desired pH is from about 4.5 to about 6.5, and the organic acid is provided in a quantity sufficient to provide such a pH. In some embodiments, the organic acid is provided in a quantity sufficient to provide a pH of from about 5.5 to about 6.5, for example, from about 5.5, about 5.6, about 5.7, about 5.8, about 5.9, or about 6.0, to about 6.1, about 6.2, about 6.3, about 6.4, or about 6.5.

In some embodiments, a mineral acid (e.g., hydrochloric acid, sulfuric acid, phosphoric acid, or the like), alone or in combination with an organic acid, is added to adjust the pH of the aerosol generating material to the desired value. In some embodiments, a buffer (e.g., a buffer as described herein below) is added to the aerosol generating material to the desired value, and/or to maintain the pH of the aerosol generating material at the desired value.

In some embodiments, the aerosol generating material further comprises a solubility enhancer to increase the solubility of one or more of the organic acid or salt thereof. Suitable solubility enhancers include, but are not limited to, humectants as described herein, such as glycerol or propylene glycol.

### Other Active Agents

In some embodiments, the aerosol precursor aerosol generating material comprises an active agent in addition to the active agents described herein above. These additional active agents include, but are not limited to, botanical extracts, cannabinoids, terpenes, tobacco extracts, and combinations thereof.

The additional active agent may be a physiologically active material, which is a material intended to achieve or enhance a physiological response. The active agent may for example be selected from nutraceuticals, nootropics, psychoactives. The active agent may be naturally occurring or synthetically obtained. The active agent may comprise for example nicotine, caffeine, taurine, theanine, vitamins such as B6 or B12 or C, melatonin, cannabinoids, or constituents, derivatives, or combinations thereof. The active agent may comprise one or more constituents, derivatives or extracts of cannabis or another botanical material or extract thereof (other than tobacco).

### Nicotine

In some embodiments, the aerosol generating material comprises nicotine as an additional active agent. In some embodiments, the aerosol generating material has a nicotine content of from about 1.5 wt % to about 7 wt % (DWB).

In some embodiments, the aerosol generating material may comprise from at least about 1.5 wt %, about 2 wt %, about 2.5 wt %, about 3 wt %, about 3.5 wt %, about 4 wt %, about 4.5 wt % or about 5 wt % of nicotine (DWB). The aerosol generating material may comprise no more than about 7 wt %, about 6.5 wt %, about 6 wt %, about 5.5 wt %, about 5 wt %, about 4.5 wt %, about 4 wt %, about 3.5 wt % or about 3 wt % of nicotine (DWB). For example, the aerosol generating material may comprise from about 2 to about 6 wt %, or from about 4 to about 5 wt % nicotine by weight of the aerosol generating material (DWB).

In some embodiments, the aerosol generating material comprises from about 1 wt %, about 1.5 wt % or about 2 wt % to about 6 wt %, about 5 wt %, about 4 wt % or about 3 wt % of nicotine (DWB).

In some embodiments, the aerosol generating material of the disclosure can be completely free or substantially free of nicotine (3-(1-methylpyrrolidin-2-yl)pyridine). In some embodiments the aerosol generating material is completely free of (R)-, (S)-, and (R/S)-3-(1-methylpyrrolidin-2-yl)pyridine (e.g., having 0% by weight of nicotine, including racemic nicotine and nicotine enantiomers, calculated as the free base and based on the total weight of the aerosol generating material).

### Cannabinoids

In some embodiments, the other active agent comprises one or more cannabinoids. As used herein, the term “cannabinoid” refers to a class of diverse natural or synthetic chemical compounds that acts on cannabinoid receptors (e.g., CB1 and CB2) in cells that alter neurotransmitter release in the brain. Cannabinoids are cyclic molecules exhibiting particular properties such as the ability to easily cross the blood-brain barrier. Cannabinoids may be naturally occurring (Phytocannabinoids) from plants such as cannabis, (endocannabinoids) from animals, or artificially manufactured (synthetic cannabinoids).

Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

In some embodiments, the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof.

In certain embodiments, the cannabinoid is selected from tetrahydrocannabinol (THC), the primary psychoactive compound in cannabis, and cannabidiol (CBD), another major constituent of the plant, but which is devoid of psychoactivity. All of the above compounds can be used in the form of an isolate from plant material or synthetically derived. Certain cannabinoids, including but not limited to CBD and THC, may exist in more than one isomeric form, for example Δ8- and Δ9-THC. Such isomeric forms may be naturally occurring or may be synthetic. For avoidance of doubt, reference within the present disclosure to a “cannabinoid” is intended to be inclusive of any and all isomeric forms thereof.

In some embodiments, the cannabinoid comprises at least tetrahydrocannabinol (THC). In some embodiments, the cannabinoid is tetrahydrocannabinol (THC). In some embodiments, the THC is Δ8-THC. In some embodiments, the THC is Δ9-THC.

In some embodiments, the cannabinoid comprises at least cannabidiol (CBD). In some embodiments, the cannabinoid is cannabidiol (CBD). In some embodiments, the CBD is synthetic CBD. In some embodiments, the CBD is Δ8-CBD. In some embodiments, the CBD is Δ9-CBD.

In some embodiments, the cannabinoid (e.g., CBD) is added to the aerosol generating material in the form of an isolate. An isolate is an extract from a plant, such as cannabis, where the active material of interest (in this case the cannabinoid, such as CBD) is present in a high degree of purity, for example greater than 95%, greater than 96%, greater than 97%, greater than 98%, or around 99% purity.

In some embodiments, the cannabinoid is an isolate of CBD in a high degree of purity, and the amount of any other cannabinoid in the aerosol generating material is no greater than about 1% by weight of the aerosol generating material, such as no greater than about 0.5% by weight of the aerosol generating material such as no greater than about 0.1% by weight of the aerosol generating aerosol generating material, such as no greater than about 0.01% by weight of the aerosol generating material.

The choice of cannabinoid and the particular percentages thereof which may be present within the aerosol generating material will vary depending upon the desired characteristics of the material.

In some embodiments, the cannabinoid (such as CBD) is present in the aerosol generating material in a concentration of at least about 0.001% by weight of the aerosol generating material, such as in a range from about 0.001% to about 2% by weight of the aerosol generating material. In some embodiments, the cannabinoid (such as CBD) is present in the aerosol generating material in a concentration of from about 0.1% to about 1.5% by weight, based on the total weight of the aerosol generating material. In some embodiments, the cannabinoid (such as CBD) is present in a concentration from about 0.4% to about 1.5% by weight, based on the total weight of the aerosol generating material.

Alternatively, or in addition to a cannabinoid, the active agent may include a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids. Such compounds can be used in the same amounts and ratios noted herein for cannabinoids.

### Terpenes

Active agents suitable for use in the aerosol generating material can also be classified as terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

In some embodiments, the terpene is a terpene derivable from a phytocannabinoid producing plant, such as a plant from the stain of the Cannabis sativa species, such as hemp. Suitable terpenes in this regard include so-called “C10” terpenes, which are those terpenes comprising 10 carbon atoms, and so-called “C15” terpenes, which are those terpenes comprising 15 carbon atoms. In some embodiments, the active agent comprises more than one terpene. For example, the active agent may comprise one, two, three, four, five, six, seven, eight, nine, ten or more terpenes as defined herein. In some embodiments, the terpene is selected from pinene (alpha and beta), geraniol, linalool, limonene, carvone, eucalyptol, menthone, iso-menthone, piperitone, myrcene, beta-bourbonene, germacrene and mixtures thereof.

Terpenes and/or cannabinoids may be present as an active agent, as an aerosol former, or as a flavorant. The amount of terpene and/or cannabinoid present may vary accordingly based on their intended purpose.

In some embodiments, the active agent comprises caffeine, melatonin, an amino acid, a vitamin, or combinations thereof. In some embodiments, the active agent comprises taurine, theanine, vitamin B6, B12, or C, melatonin, cannabinoids, or constituents, derivatives, or combinations thereof.

### Filler

In some embodiments, the aerosol generating material further comprises a filler. The filler may be a non-tobacco component, that is, a component that does not include ingredients or components originating from tobacco. The filler may comprise one or more inorganic filler materials, such as calcium carbonate, perlite, vermiculite, diatomaceous earth, colloidal silica, magnesium oxide, magnesium sulphate, magnesium carbonate, and suitable inorganic sorbents, such as molecular sieves. The filler may be a non-tobacco fiber such as wood fiber or pulp or wheat fiber. The filler can be a material comprising cellulose or a material comprises a derivate of cellulose. The filler component may also be a non-tobacco cast material or a non-tobacco extruded material. In some embodiments, the filler is cellulosic material, cellulose or CMC. In some embodiments, the filler is essentially composed or consists of cellulose.

In particular embodiments which include filler, the filler is fibrous. For example, the filler may be a fibrous organic filler material such as wood, wood pulp, hemp fiber, cellulose or cellulose derivatives. Without wishing to be bound by theory, it is believed that including a fibrous filler may increase the tensile strength of the aerosol generating material. The use of cellulose as a filler may have a particularly favorable impact on the burst strength of the aerosol generating material. The filler may also contribute to the texture of the aerosol generating material. For example, a fibrous filler, such as cellulose, may provide an aerosol generating material having relatively rough first and second surfaces. Conversely, a non-fibrous, particulate filler, such as powdered chalk, may provide an aerosol generating material having relatively smooth first and second surfaces. In some embodiments, the aerosol generating material comprises a combination of different filler materials. The filler may help to improve the general structural properties of the aerosol generating material, such as its tensile strength and burst strength.

The total amount of filler in the aerosol generating material may vary. In some embodiments, the total amount of filler is at least about 1% by weight or at least about 5% by weight or at least about 10% by weight, based on the total weight of aerosol generating material. In some embodiments, the filler is present in an amount of about 1% by weight to about 40% by weight, such as about 2% to about 30% by weight.

### Flavoring Agent

In some embodiments, the aerosol generating material as described herein comprises a flavoring agent. As used herein, a “flavoring agent” or “flavorant” is any flavorful or aromatic substance capable of altering the sensory characteristics associated with the aerosol generating material or the vapor produced therefrom. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy.

Flavoring agents may be imitation, synthetic or natural ingredients or blends thereof. Flavoring agents may include naturally occurring flavor materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents.

Flavorants may further include flavor enhancers, sensorial receptor site activators or stimulators, and trigeminal sensates, As used herein, “trigeminal sensate” refers to a flavoring agent which has an effect on the trigeminal nerve, producing sensations including heating, cooling, tingling, and the like. Non-limiting examples of trigeminal sensate flavoring agents include capsaicin, citric acid, menthol, Sichuan buttons, erythritol, and cubebol.

In some embodiments, the aerosol generating material comprises a sensate which provides to the user of such aerosol generating material a cooling effect. Suitable cooling agents include, but are not limited to, menthane, menthone, menthone ketals, menthone glycerol ketals, substituted p-menthanes, acyclic carboxamides, monomenthyl glutarate, substituted cyclohexanamides, substituted cyclohexane carboxamides, substituted ureas and sulfonamides, substituted menthanols, hydroxymethyl and hydroxymethyl derivatives of p-menthane, 2-mercapto-cyclo-decanone, hydroxycarboxylic acids with 2-6 carbon atoms, cyclohexanamides, menthyl acetate, menthyl salicylate, N-ethyl-p-menthane-3-carboxamide (WS-3), ethyl ester of N-[[5-methyl-2-(1-methylethyl)cyclohexyl]carbonyl]glycine (WS-5), WS-14, N,2,3-trimethyl-2-isopropyl butanamide (WS-23), WS-27, WS-30, (−)-Menthyloxyethanol (Coolact® 5), WS-NA (FEMA4693), WS-116 (FEMA4603), N-ethyl-2,2-diisopropylbutanamide, isopulegol, menthyloxy propane diol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxaspiro[4,5]decane-2-methanol, menthyl succinate and its alkaline earth metal salts, trimethylcyclohexanol, N-ethyl-2-isopropyl-5-methylcyclohexanecarboxamide, Japanese mint oil, peppermint oil, 3-(1-menthoxy)ethan-1-ol, 3-(1-menthoxy)propan-1-ol, 3-(1-menthoxy)butan-1-ol, 1-menthylacetic acid N-ethylamide, 1-menthyl-4-hydroxypentanoate, 1-menthyl-3-hydroxybutyrate, menthyl glutarate, N,2,3-trimethyl-2-(1-methylethyl)-butanamide, N-ethyl-trans-2-cis-6-nonadienamide, N,N-dimethyl menthyl succinamide, N-(2-hydroxyethyl)-2,3-dimethyl-2-isopropylbutanamide, substituted p-menthanes, substituted p-menthane-carboxamides, 2-isopropanyl-5-methylcyclohexanol, menthyl ethylene glycol carbonate, menthone glycerol ketals (e.g., menthone 1,2-glycerol ketal), menthone (S)-lactic acid ketal, menthyl acetoacetate, 3-1-menthoxypropane-1,2-diol, menthyl lactate, eucalyptus extract, menthol propylene glycol carbonate, menthol ethylene glycol carbonate, menthol glyceryl ether, N-tert-butyl-p-menthane-3-carboxamide, p-menthane-3-carboxylic acid glycerol ester, methyl-2-isopropyl-bicyclo[2.2.1]heptane-2-carboxamide, (1R,2S,5R)—N-(4-(carbamoylmethyl)phenyl)-menthylcarboxamide, 2-[2-(p-menthan-3-yloxy)ethoxy]ethanol, (1R,2R,4R)-1-(2-Hydroxy-4-methylcyclohexyl)ethenone, 2-(p-tolyloxy)-N-(1H-pyrazol-5-yl)-N-((thiophen-2-yl)methyl)acetamide, menthol methyl ether, menthyl pyrrolidone carboxylate, 2,5-dimethyl-4-(1-pyrrolidinyl)-3(2H)-furanone, cyclic a-keto enamines, and cyclotene derivatives (e.g., 3-methyl-2-(1-pyrrolidinyl)-2-cyclopenten-1-one and 5-methyl-2-(1-pyrrolidinyl)-2-cyclopenten-1-one). Other compounds include the alpha-keto enamines disclosed in U.S. Pat. No. 6,592,884 to Hofmann et al., which is incorporated in its entirety herein. These and other suitable cooling agents are further described in the following U.S. patents, all of which are incorporated in their entirety by reference hereto: U.S. Pat. Nos. 4,230,688; 4,032,661; 4,459,425; 4,178,459; 4,296,255; 4,136,163; 5,009,893; 5,266,592; 5,698,181; 6,277,385; 6,627,233; 7,030,273. Still other suitable cooling agents are further described in US Patent Application Publications Nos. 2005/0222256 and 2005/0265930, each of which are incorporated in their entirety by reference hereto. In some embodiments, the cooling agent comprises menthol, eucalyptus, mint, menthol, menthyl esters, eucolyptol, WS-3, WS-23, WS-5, (1R,2S,5R)—N-(4-(cyanomethyl)phenyl)menthylcarboxamide (Evercool™ 180), (1R,2S,5R)—N-(2-(pyridin-2-yl)ethyl)menthylcarboxamide (Evercool™ 190), or a combination thereof.

In some embodiments, the aerosol generating material does not comprise a flavoring agent, and comprises only a cooling agent to provide the desired user experience. In some embodiments, the cooling agent is WS-3.

In some embodiments, the aerosol generating material comprises a modulator or sensate which provides to the user of such aerosol generating material a warming effect. Suitable warming agents include, but are not limited to, ethers of vanillyl alcohol (e.g., methyl, ethyl, n-propyl, isopropyl, n-butyl, isobutyl, isoamyl, n-hexyl), gingerol, shogaol, paradol, zingerone, capsaicin, dihydrocapsaicin, nordihydrocapsaicin, homocapsaicin, homodihydrocapsaicin, benzyl alcohol, and combinations thereof. In some embodiments, the warming agent comprises vanillyl butyl ether, vanillyl ethyl ether, capsaicin, or a combination thereof.

Flavoring agents may be in any suitable form, for example, a liquid such as an oil, or a solid such as a powder or wax. In some instances, the flavoring agent may be provided in a spray-dried form or a liquid form.

The amount of flavoring agent utilized in the aerosol generating material can vary, but is typically up to about 60% by weight. For example, aerosol generating material may comprise up to about 60 wt %, about 50 wt %, about 40 wt %, about 30 wt %, about 20 wt %, about 10 wt % or about 5 wt % of a flavoring agent. In some embodiments, the aerosol generating material may comprise at least about 0.5 wt %, about 1 wt %, about 2 wt %, about 5 wt %, about 10 wt %, about 20 wt % or about 30 wt % of flavoring agent (calculated on a dry weight basis). For example, the aerosol generating material may comprise from about 10 to about 60 wt %, from about 20 to about 50 wt % or from about 30 to about 40 wt % of flavoring agent.

In some embodiments, the flavor comprises menthol, spearmint and/or peppermint.

In some embodiments, the flavor comprises flavor components of cucumber, blueberry, citrus fruits and/or redberry.

In some embodiments, the flavor comprises eugenol.

In some embodiments, the flavoring agent (if present) comprises, consists essentially of, or consists of, menthol. In some embodiments, the aerosol generating material does not comprise an added flavor.

### Water

In some embodiments, the aerosol generating material comprises water. The water content of the aerosol generating material described herein may vary according to, for example, the temperature, pressure and humidity conditions at which the material is maintained. The water content can be determined by Karl-Fisher analysis, as known to those skilled in the art. In some embodiments, the aerosol generating material comprises water in an amount of less than about 20%, less than about 15%, less than about 10%, less than about 5% by weight, or less than 2.5% by weight of the aerosol generating material. In some embodiments, the aerosol generating material comprises water in an amount of between about 0% and about 15% or between about 1% and about 10% by weight of the aerosol generating material. In some embodiments, the aerosol generating material comprises water in an amount of between about 1% and about 5% by weight of the aerosol generating material. The aerosol generating material therefore has a lower water content than the mixture of the first composition and the second composition.

The aerosol generating material may comprise total volatiles in an amount of less than about 40%, less than about 30%, less than about 20%, less than about 15%, less than about 10% or less than about 5% by weight of the aerosol generating material. Unless otherwise stated, as used herein, the phrases “volatile components”, “volatiles”, “total volatile”, “volatile content” and “total volatiles” are used to refer to volatile compounds, including water. The volatile content of a material may be measured as the reduction in mass when a sample is dried in a forced draft oven at a temperature regulated to 110° C.±1° C. for three hours±0.5 minutes. After drying, the sample is cooled in a desiccator to room temperature for approximately 30 minutes, to allow the sample to cool. In some embodiments, the aerosol generating material comprises total volatiles in an amount of between about 0% and about 40%, between about 10% and about 35% or between about 20% and about 30% by weight of the aerosol generating material. In some embodiments, the aerosol generating material may have a total volatile content of about 25% to about 35% by weight.

### Functional Materials

In some embodiments, the aerosol generating material comprises one or more functional materials. The one or more other functional materials may comprise one or more of pH regulators, coloring agents, preservatives, thickeners (e.g., glucomannan), emulsifiers, stabilizers, and/or antioxidants.

### Format of Aerosol Generating Material

The form of the aerosol generating material may vary. For example, the aerosol generating material may be present in an article as disclosed herein in sheet form, shredded form, bead form, fibrous form, or extrudate form.

In some embodiments, the aerosol generating material is in sheet form or shredded sheet form, said sheet or shredded sheet having a first and second surface. The first and/or second surfaces of the sheet or shredded sheet may be relatively uniform (e.g., they may be relatively smooth) or they may be uneven or irregular. For example, the first and/or second surfaces of the sheet may be textured or patterned to define a relatively coarse surface. In some embodiments, the first and/or second surfaces are relatively rough.

The smoothness of the first and second surfaces may be influenced by a number of factors, such as the area density of the sheet or shredded sheet, the nature of the components that make up the aerosol-generating material or whether the surfaces of the material have been manipulated, for example embossed, scored or otherwise altered to confer them with a pattern or texture.

The thickness of the sheet or shredded sheet may vary. If the sheet or shredded sheet of aerosol-generating material is too thick, then heating efficiency can be compromised. This can adversely affect power consumption in use, for instance the power consumption for release of flavor and/or active agent from the aerosol generating material. Conversely, if the sheet or shredded sheet of aerosol generating material is too thin, it can be difficult to manufacture and handle; a very thin material can be harder to cast and may be fragile, compromising aerosol formation in use. The thickness of a sheet can be determined using ISO 534:2011 “Paper and Board-Determination of Thickness”.

In some embodiments, the sheet or shredded sheet of aerosol generating material has a thickness of at least about 100 μm, such as at least about 100 μm, 120 μm, 140 μm, 160 μm, 180 μm, 200 μm, 220 μm, 240 μm, 260 μm, 280 μm, 290 μm, or 300 μm. In some embodiments, the sheet or shredded sheet has a thickness of from about 100 μm to about 300 μm, from about 151 μm to about 299 μm, from about 152 μm to about 298 μm, from about 153 μm to about 297 μm, from about 154 μm to about 296 μm, from about 155 μm to about 295 μm, from about 156 μm to about 294 μm, from about 157 μm to about 293 μm, from about 158 μm to about 292 μm, from about 159 μm to about 291 μm or from about 160 μm to about 290 μm. In some embodiments, the sheet or shredded sheet has a thickness of from about 170 μm to about 280 μm, from about 180 to about 270 μm, from about 190 to about 260 μm, from about 200 μm to about 250 μm or from about 210 μm to about 240 μm. In some embodiments, the thickness of the sheet or shredded sheet is about 230 μm to about 270 μm or about 240 μm to about 260 μm.

The thickness of the sheet or shredded sheet may vary between the first and second surfaces. In some embodiments, an individual strip or piece of the aerosol generating material has a minimum thickness over its area of about 100 μm. In some embodiments, an individual strip or piece of the sheet or shredded sheet of aerosol-generating material has a minimum thickness over its area of about 0.05 mm or about 0.1 mm. In some embodiments, an individual strip, strand or piece of the sheet or shredded sheet of aerosol generating material has a maximum thickness over its area of about 1.0 mm. In some embodiments, an individual strip or piece of the aerosol generating material has a maximum thickness over its area of about 0.5 mm or about 0.3 mm.

It is postulated that if the sheet or shredded sheet of aerosol generating material is too thin (e.g. less than 100 μm), then it may be necessary to increase the cut width of the shredded sheet to achieve sufficient packing of the sheet or shredded sheet of aerosol generating material when it is incorporated into an article. Increasing the cut width of the shredded sheet can increase the pressure drop, which is undesirable.

The thickness of the sheet or shredded sheet is also thought to have a bearing on its area density. That is to say, increasing the thickness of the sheet or shredded sheet may increase the area density of the sheet or shredded sheet. Conversely, decreasing the thickness of the sheet or shredded sheet may decrease the area density of the sheet or shredded sheet. For the avoidance of doubt, where reference is made herein to area density, this refers to an average area density calculated for a given strip, strand, piece or sheet of the aerosol-generating material, the area density calculated by measuring the surface area and weight of the given strip, strand, piece or sheet of aerosol-generating material.

It is postulated that an aerosol generating material having a thickness of at least about 100 μm, along with an area density of from about 100 g/m² to about 240 or 250 g/m² is less liable to tear, split or become otherwise deformed during its manufacture. For example, it may have a good tensile strength and thus be relatively easy to process. In some embodiments, the area density is between about 170 and about 240 or 250 g/m². In some embodiments, the area density is about 180 g/m². In some embodiments, the sheet or shredded sheet of aerosol generating material has an area density of from about 100 g/m² to about 250 g/m², such as from about 110 g/m² to about 240 g/m², from about 120 g/m² to about 230 g/m², from about 130 g/m² to about 220 g/m² or from about 140 g/m² to about 210 g/m². In some embodiments, the sheet or shredded sheet has an area density of from about 130 g/m² to about 190 g/m², from about 140 g/m² to about 180 g/m², from about 150 g/m² to about 170 g/m². In some embodiments, the sheet or shredded sheet has an area density of about 180 g/m².

The area density of about 100 g/m² to about 250 g/m² is thought to contribute to the strength and flexibility of sheet or shredded sheet. Furthermore, a rod comprising a shredded sheet of aerosol generating material having an area density of around 180 g/m² and a minimum thickness of 220-230 μm can be packed such that the aerosol generating material stays in place within the rod whilst maintaining a desired weight of material within the rod (e.g. around 300 mg) and delivering acceptable organoleptic properties (e.g. taste and smell) when heated in a non-combustible aerosol provision device.

The flexibility of the sheet or shredded sheet is considered to be dependent, at least in part, upon the thickness and area density of the sheet or shredded sheet. A thicker sheet or shredded sheet may be less flexible than a thinner sheet or shredded sheet. Also, the greater the area density of the sheet, the less flexible the sheet or shredded sheet is. It is thought that the combined thickness and area density of the aerosol-generating material described herein provides a sheet or shredded sheet that is relatively flexible. When the aerosol generating material is incorporated into an article for use in a non-combustible aerosol-provision device, this flexibility, may give rise to various advantages. For example, the strands or strips are able to readily deform and flex when an aerosol generator (e.g. a heater) is inserted into the aerosol generating material, thus facilitating insertion of an aerosol generator into the material and also improving retention of the aerosol generator by the aerosol generating material.

The area density of the sheet or shredded sheet of aerosol-generating material may influence the roughness of the first and second surfaces of the sheet or shredded sheet. By changing the area density, the roughness of the first and/or second surfaces can be tailored.

In some embodiments, the sheet or shredded sheet may have a tensile strength of at least 3 N/15 mm or at least around 4 N/15 mm. Where the sheet or shredded sheet has a tensile strength below 3 N/15 mm, the sheet or shredded sheet is likely to tear, break or otherwise deform during its manufacture and/or subsequent incorporation into an article for use in a non-combustible aerosol provision system. Tensile strength may be measured using ISO 1924:2008.

The sheet or shredded sheet of aerosol generating material may have a burst strength of at least about 75 g, at least about 100 g or at least about 200 g. In some embodiments, the burst strength of the sheet or shredded sheet of aerosol-generating material is at least 150 g. As disclosed and discussed above, the burst strength affects the strength of the material.

In some embodiments, the aerosol generating material is present in the form of a continuous sheet which is optionally crimped and gathered. In some embodiments, the continuous sheet in gathered form comprises a region configured to receive a heater (referred to herein as an aerosol generator). For example, the gathered sheet may comprise a recess to accommodate a heater. The gathered sheet may further comprise passages through the sheet to allow air to pass through the gathered sheet.

In some embodiments, the aerosol generating material is present in an article in the form of an extruded solid plug. In some embodiments, the extruded solid plug comprises a region configured to receive a heater (referred to herein as an aerosol generator). For example, the extruded plug may comprise a recess to accommodate a heater. The recess or cavity can extend partially or completely through the extruded plug. The extruded plug may further comprise passages through the plug to allow air to pass through the plug.

In some embodiments, the aerosol generating material is present in an article as disclosed herein in bead form, such as a plurality of beads. For example, the material or a composition utilized to form the material as described herein below, may be extruded and formed into beads using conventional techniques.

In some embodiments, the aerosol generating material in extruded form (e.g., as a continuous sheet which may be crimped and gathered, in shredded sheet form, as an extruded plug, or as beads) comprises a particulate botanical material, a filler, a binder, an aerosol former, and an active agent, each as described herein. In some embodiments, the aerosol generating material comprises from about 50% to about 90% by weight of the particulate botanical material, from about 1% to about 7% by weight of the filler, from about 1% to about 20% by weight of the binder, and from about 5% to about 30% by weight of the aerosol former. In some embodiments, the particulate botanical material is rooibos. In some embodiments, the filler is a cellulosic material such as microcrystalline cellulose, wood pulp, or a combination thereof. In some embodiments, the binder is a cellulosic material, such as a cellulose ether. In some embodiments, the binder is a polysaccharide or alginate.

In some embodiments, the aerosol generating material is in fibrous form. As used herein, the term “fiber” is defined as a basic element of textiles. Fibers are often in the form of a rope- or string-like element. As used herein, the term “fiber” is intended to include fibers, filaments, continuous filaments, staple fibers, and the like, which can be monocomponent or multicomponent. The term “multicomponent fibers” refers to fibers that comprise two or more components that are different by physical or chemical nature, including bicomponent fibers. Specifically, the term “multicomponent fibers” includes staple and continuous fibers prepared from two or more polymers present in discrete structured domains in the fiber, as opposed to blends where the domains tend to be dispersed, random or unstructured.

In particular, the fibrous form can be a woven or nonwoven material. The term “nonwoven” is used herein in reference to fibrous materials, webs, mats, batts, or sheets in which fibers are aligned in an undefined or random orientation. The nonwoven fibers are initially presented as unbound fibers or filaments. An important step in the manufacturing of nonwovens involves binding the various fibers or filaments together. The manner in which the fibers or filaments are bound can vary, and include thermal, mechanical and chemical techniques that are selected in part based on the desired characteristics of the final product.

Example materials of the fibers may include, but are not limited to, a polymer selected from the group consisting of polyglycolic acid, polylactic acid, polyhydroxyalkanoates, polycaprolactone, polybutylene succinate, polybutylene succinate adipate, and copolymers thereof. In some embodiments, the fibers may be selected from the groups consisting of wool, cotton, fibers made of cellulosic material (such as regenerated cellulose, cellulose acetate, cellulose triacetate, cellulose nitrate, ethyl cellulose, cellulose acetate propionate, cellulose acetate butyrate, hydroxypropyl cellulose, methyl hydroxypropyl cellulose), protein fibers, and the like.

Regenerated cellulose fibers can be particularly advantageous, and are typically prepared by extracting non-cellulosic compounds from wood, contacting the extracted wood with caustic soda, followed by carbon disulfide and then by sodium hydroxide, giving a viscous solution. The solution is subsequently forced through spinneret heads to create viscous threads of regenerated fibers. Example methods for the preparation of regenerated cellulose are provided in U.S. Pat. No. 4,237,274 to Leoni et al; U.S. Pat. No. 4,268,666 to Baldini et al; U.S. Pat. No. 4,252,766 to Baldini et al.; U.S. Pat. No. 4,388,256 to Ishida et al.; U.S. Pat. No. 4,535,028 to Yokogi et al.; U.S. Pat. No. 5,441,689 to Laity; U.S. Pat. No. 5,997,790 to Vos et al.; and U.S. Pat. No. 8,177,938 to Sumnicht, which are incorporated herein by reference. The manner in which the regenerated cellulose is made is not limiting, and can include, for example, both the rayon and the TENCEL processes. Various suppliers of regenerated cellulose are known, including Lenzing (Austria), Cordenka (Germany), Aditya Birla (India), and Daicel (Japan).

The form of the fibers used in the nonwoven web according to the present disclosure can vary, and include fibers having any type of cross-section, including, but not limited to, circular, rectangular, square, oval, triangular, and multilobal. In certain embodiments, the fibers can have one or more void spaces, wherein the void spaces can have, for example, circular, rectangular, square, oval, triangular, or multilobal cross-sections. As noted previously, the fibers can be selected from single-component (i.e., uniform in composition throughout the fiber) or multicomponent fiber types including, but not limited to, fibers having a sheath/core structure and fibers having an islands-in-the-sea structure, as well as fibers having a side-by-side, segmented pie, segmented cross, segmented ribbon, or tipped multilobal cross-sections.

The physical parameters of the fibers present in the nonwoven web can vary. For example, the fibers used in the nonwoven web can have varying size (e.g., length, denier per filament (dpf)) and crimp characteristics. In some embodiments, fibers used in the nonwoven web can be nano fibers, sub-micron fibers, and/or micron-sized fibers. In certain embodiments, fibers of the nonwoven webs useful herein can measure about 1.5 dpf to about 5.0 dpf, or about 1.6 dpf to about 3.0 dpf. In various embodiments, each fiber can measure about 4-10 crimps per cm, or about 5-8 crimps per cm. In some embodiments, each fiber can be a continuous filament fiber. In certain embodiments, each fiber can be a staple fiber. Each fiber length can measure about 35 mm to about 60 mm, or about 38 mm to about 55 mm, for example. It can be advantageous for all fibers in the nonwoven web to have similar fiber size and crimp attributes to ensure favorable blending and orientation of the fibers in the nonwoven web.

### Process of Making Aerosol Generating Material

In another aspect is provided a process for preparing an aerosol generating material as disclosed herein. The process generally comprises forming a first composition comprising a binder and optionally an aerosol former; forming a second composition comprising a non-tobacco botanical material and a filler; combining the first composition and the second composition to form a mixture of the first composition and the second composition; and processing the mixture of the first composition and the second composition to form the aerosol generating material. Each of the binder, aerosol former, non-tobacco botanical, filler, and aerosol generating material are as described herein above. In some embodiments, the first and/or the second composition may comprise an active agent as described herein (e.g., a substituted 3-(1-methylpyrrolidin-2-yl)pyridine as described herein). In some embodiments, the first and/or the second composition are completely free of substituted 3-(1-methylpyrrolidin-2-yl)pyridine. The first composition may comprise water. Water may also be present in the non-tobacco botanical material and/or the filler.

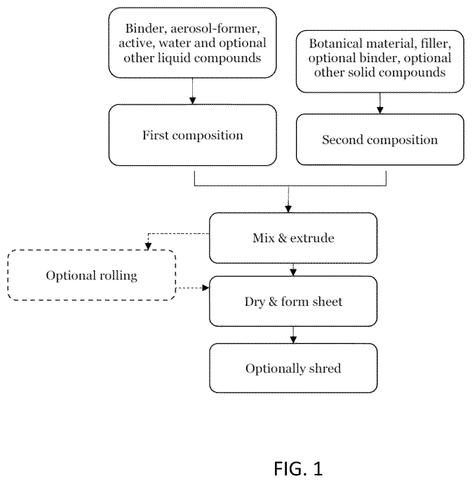

FIG. 1 is a non-limiting illustration of how an aerosol generating material according to embodiments of the disclosure may be manufactured. With reference to FIG. 1, a first composition comprising a binder, optionally an active agent, water and an aerosol former and a second composition comprising non-tobacco botanical material, filler, and optionally a second binder are formed and mixed. In the subsequent step, the first composition and the second composition are mixed and extruded through a suitable die. The extrudate can be shaped into any desired form, such as a cylindrical rod, which may be cut to the desired length. In some embodiments, the extruded material forms a solid plug of aerosol generating material. In some embodiments, the extruded material comprises a central cavity configured to receive a pin heater.

Following the extrusion, the extruded mixture of the first composition and the second composition may be dried to form a sheet of aerosol generating material. In an optional step, the extruded mixture of the first composition and the second composition may be rolled to form a sheet prior to drying the sheet. The sheet may then be shredded in order to make the aerosol-generating material, which may then be incorporated into a consumable for a non-combustible aerosol delivery system. The sheet may be shredded to form a plurality of strands or strips of aerosol generating material. The sheet may be crimped and gathered.

The first composition, also known as the “wet mixture”, may comprises an aerosol former or humectant, optionally an active agent, water and a binder, each as described herein above. The wet mixture may be in the form of a suspension. The first composition may also comprise other liquids or suspensions disclosed herein. The first composition may be in a liquid phase. The first composition comprises a binder as described herein above. The binder is arranged to bind the components of the first composition. Once combined with the second composition, the binder binds the components of the first and second compositions to form the aerosol generating material. The first composition can comprise more than one binder. In such embodiments, the binders in the first composition can be the same or different.

The second composition, also referred to herein as the “dry mixture”, comprises a non-tobacco botanical material, a filler and optionally a second binder, each as described herein above. The second composition may also comprise other solids or gels disclosed herein. The second composition may be in the solid phase.

Incorporating relatively high quantities of the binder relative to the aerosol former in the first composition can result in a highly viscous mixture and, as a consequence, difficulties with blending the first composition with the second composition. This problem can be solved by reducing the amount of binder in the first composition and adding a second binder (which can be the same or different to the first binder) to the second composition. Therefore, the second composition may comprise an optional second binder. In some embodiments, the first and the second binders are the same. In some embodiments, the first and the second binders are the different. The binder may be selected from one or more compounds selected from the group comprising alginates, pectins, starches (and derivatives), celluloses (and derivatives), gums, silica or silicones compounds, clays, polyvinyl alcohol and combinations thereof. For example, in some embodiments, the binder comprises one or more of alginates, pectins, hydroxyethyl cellulose, hydroxypropyl cellulose, carboxymethylcellulose (CMC), pullulan, xanthan gum, guar gum, carrageenan, agarose, acacia gum, fumed silica, PDMS, sodium silicate, kaolin and polyvinyl alcohol. In some cases, the binder comprises alginate and/or pectin or carrageenan. In some embodiments, the binder comprises CMC.

By incorporating the second binder into the second composition, the amount of first binder in the first composition can be reduced, thus lowering the viscosity of the first composition and facilitating the formation of a mixture of the first composition and the second composition. The binder may at least partially coat the surface of the non-tobacco botanical material. Where the botanical material is in a particulate form, the binder may at least partially coat the surface of the particles of botanical and bind them together.

The total volatile content of the second composition may be about 5%-40% or about 10-20% by weight of the second composition. The total volatile content of the second composition may be about 5%, 10%, 15%, 20%, 25%, 30%, 35% or 40% by weight of the second composition.

The first and the second compositions described herein may be mixed to provide a mixture of the first composition and the second composition. The mixture of the first composition and the second composition may be formed by homogenizing the first composition and the second composition. The mixture of the first composition and the second composition may be in the form of a “dough”. Water may be included in the first composition. The inclusion of water in the first composition helps to ensure a homogeneous dispersion of the binder throughout the mixture. The water may also assist with the hydration of the binder.

The aerosol generating material produced by the process described herein exhibits homogeneous properties and thus the aerosol that is produced by the aerosol generating material is relatively consistent. By forming the first composition comprising a binder and optionally an aerosol former separately from the second composition comprising a non-tobacco botanical material and a filler and then combining the first composition and the second composition to form a mixture of the first composition and the second composition, a homogeneous aerosol generating material is formed, where the non-tobacco botanical material, filler, binder and active are evenly distributed throughout the aerosol generating material.

In some embodiments, minimal water, or no water at all, may be required to be added to the mixture to provide a homogenous dough that is suitable for subsequent processing steps. For example, the dough may then be extruded via a die, through which a homogenous dough may suitably pass without the further addition of water or the addition of a small amount of water.

In some embodiments, the first composition comprises water. The inclusion of water in the first composition hydrates the binder and assists with the formation of a homogenous mixture.

Mixing the first binder, optional active, optional aerosol former, non-tobacco botanical material, filler and optional second binder in a single step (i.e., without forming the first and second compositions separately and then combining them) can result in a viscous mixture that is difficult to process and handle. By forming the first composition and second composition separately and then combining these compositions, the resultant “dough”-like mixture can be more easily processed.

The non-tobacco botanical material may be present in an amount of from about 10% to about 90% by weight of the mixture of the first composition and the second composition. For example, the botanical material may be present in an amount of about 10%, 20%, 30%, 40%, 50%, 60%, 70%, 80%, or 90% by weight of the mixture of the first composition and the second composition. In some embodiments, the non-tobacco botanical material is present in an amount of about 10% to 80% or from about 20% to 50% by weight of the mixture of the first composition and the second composition. The non-tobacco botanical material may be present in an amount of about 21%, 22%, 23%, 24%, 25%, 26%, 27%, 28%, 29%, 30%, 31%, 32%, 33%, 34%, 35%, 36%, 37%, 38%, 39% or 40%.

The filler component may be present in an amount of 0 to 20% by weight of the mixture of the first composition and the second composition, or in an amount of from 1 to 10% by weight of the mixture of the first composition and the second composition. For example, the filler may be present in an amount of greater than about 1%, 2%, 3%, 4%, 5%, 6%, 7%, 8%, 9% or 10% by weight of the mixture of the first composition and the second composition. In some embodiments, the filler component is present in an amount of 5 to 10% by weight of the mixture of the first composition and the second composition. The inclusion of 5 to 10% of the filler may improve the burst strength and reduce the brittle nature of the aerosol-generating material.

The aerosol former may be present in an amount of from about 10% to about 25% by weight of the mixture of the first composition and the second composition, or in an amount of from 1 to about 10% by weight of the mixture of the first composition and the second composition. For example, aerosol former may be present in an amount of about 10%, 12%, 15%, 18%, 20%, or 25% by weight of the mixture of the first composition and the second composition. In some embodiments, aerosol former is present in an amount of about 15%, 16%, 17%, 18% or 19% by weight of the mixture of the first composition and the second composition.

The binder may be present in an amount of from about 1 to about 20% by weight of the mixture of the first composition and the second composition, or in an amount of from 1 to about 10% by weight of the mixture of the first composition and the second composition. For example, the binder may be present in an amount of greater than about 1%, 2%, 3%, 4%, 5%, 6%, 7%, 8%, 9% or 10% by weight of the mixture of the first composition and the second composition.

In some embodiments, the binder is present in an amount of greater than about 2% by weight of the mixture of the first composition and the second composition. In some embodiments, the binder is present in an amount of about, or up to about, 5% by weight of the mixture of the first composition and the second composition. The amount of binder in the first composition, second composition and mixture of the first and second compositions is important as this alters the consistency of the compositions and mixtures. Too much binder may cause the composition/mixture to be too viscous to be processed, for example by pumps and machinery.

In some embodiments, there is provided a first and a second binder, and the ratio of the first binder to the second binder is between 1:1 and 1:10. This is advantageously selected to maintain the physical properties of the sheet and to provide adequate binding of the mixture and/or the aerosol generating material without altering the texture of the composition negatively. The ratio between the first and the second binder may be about 1:10, 2:8, 3:7, 4:6, 5:5, 10:1, 8:2, 7:3, 6:4 respectively. In some embodiments, the ratio between the first binder to the second binder is 4:6 respectively to maintain the physical properties sheet or shredded sheet. Including all the binder in the first composition may make the first composition become too viscous to be processed, for example by pumps and machinery. Providing the binder in both the first composition and the second composition makes the compositions easier to process.

The incorporation of the first binder, the optional second binder and the filler in a total amount of between about 1% and about 15% by weight of the mixture of the first composition and the second composition may have a beneficial effect on the burst strength, strength and flexibility of the aerosol generating material. The mixture of the first composition and the second composition may comprise about 2%, about 5%, about 8%, about 10%, about 12% or about 15% on a dry weight basis (dwb) in total of the first binder, the optional second binder and the filler. In some embodiments, the mixture of the first composition and the second composition comprises 5% the first binder and the optional second binder and 5% the filler. This incorporation of the first binder, the optional second binder and the filler may decrease the tackiness, increase its burst strength and improve flexibility of the aerosol-generating material.

In a particular embodiment, the first and optional second binder are both CMC, the total amount of binder is 5%, the filler is cellulose and the total amount of cellulose is 8.2%. Thus, in such an embodiment, the mixture of the first composition and the second composition comprises 5% CMC and 8.2% cellulose.

In some embodiments, the first and optional second binder are both CMC, the total amount of binder is 10%, the filler is cellulose and the total amount of cellulose is 14%. Thus, in such an embodiment, the mixture of the first composition and the second composition comprises 10% CMC and 14% cellulose.

The presence of the binder and the filler in these amounts may have a particularly beneficial effect on the physical properties of the aerosol generating material, including improved strength and flexibility. Cellulose filler improves the burst strength and reduces the brittleness of the aerosol generating material. The burst strength of the aerosol generating material produced by the process described herein can be measured using a calibrated Texture Analyser (50 kg load cell, 20 mm probe height calibration, 1 g contact force) and Exponent software, made by Stable Micro Systems. The burst strength can be determined using a 3 cm² sheet of aerosol generating material using a 5 mm stainless steel ball probe. Burst strength can be reported in units Force (g). The aerosol generating material may have a burst strength of at least about 75 g, at least about 100 g or at least about 200 g. In some embodiments the aerosol generating material may have a burst strength of at least 150 g.

If the burst strength is too low, the aerosol generating material may be relatively brittle. As discussed herein, the aerosol generating material may be formed into a sheet or a shredded sheet. As a consequence, breakages in the sheet or shredded sheet may occur during the process of manufacturing the aerosol generating material. For example, when the sheet is shredded to form a shredded sheet by a cutting process, the sheet may shatter or break into pieces or shards when cut. The incorporation of the first binder, the optional second binder and the filler may help to improve the general structural properties of the aerosol generating material, such as its tensile strength and burst strength.

The total volatile content (oven volatiles) of the mixture of the first and second compositions may be greater than 20% by weight of the mixture of the first composition and the second composition. The volatile content may be greater than about 5%, 10%, 15%, 20%, 25%, 30%, 40%, 50% or 60% by weight of the mixture of the first composition and the second composition. In some embodiments, about 20 to about 60% water is added to the mixture of the first composition and the second composition. In some embodiments, about 30% to 60% or 40% to 60% water is added.

The total volatile content of the mixture of the first composition and the second composition may be about % by weight. The total volatile content may be about 5%, 10%, 15%, 20%, 25%, 30%, 40%, 50%, 60%, 70% or 80% by weight of the mixture of the first composition and the second composition.

The water content of the mixture of the first and second compositions may be greater than 20% by weight of the mixture of the first composition and the second composition. The water content may be greater than about 5%, 10%, 15%, 20%, 25%, 30%, 40%, 50% or 605 by weight of the mixture of the first composition and the second composition. In some embodiments, about 20 to about 60% water is added to the mixture of the first composition and the second composition. In some embodiments, about 30% to 60% or 40% to 60% water is added. The total water content of the mixture of the first composition and the second composition may be about 40 to 90% by weight. The total water content may be about 5%, 10%, 15%, 20%, 25%, 30%, 40%, 50%, 60%, 70%, 80% or 90% by weight of the mixture of the first composition and the second composition.

In some embodiments, and additional advantage of the process is that less water is required to make the dough-like mixture of the first composition and the second composition that in other traditional compositions. This has the advantage that the mixture of the first composition and the second composition can therefore be easily mixed without the need to add additional water or agents to form a homogenous mix that is suitable for extrusion. An additional benefit of the lower amount of water necessary is that this improves the reliability of the manufacturing process. As a consequence, the manufacturing process is also repeatable, which also has cost-saving implications. The total water content of the mixture of the first composition and the second composition is therefore relatively low.

As a consequence of this lower water content, less water is required to be removed during the processing stages. For example, a “bandcasting” process uses a slurry of which the target water content is about 75% to about 80%. This slurry must then be dried to a target water content of about 13%, therefore requiring a loss of about 67% of water. In the present disclosure, minimal water is incorporated into the mixture of the first composition and the second composition. For example, from the resultant dough to the final product, there may be a loss of only about 47% of water. Therefore, the water loss may be significantly lower in the process described herein compared to methods to form aerosol-generating materials that comprise a slurry such as a bandcasting process. Advantageously, as less water is required to be removed during the processing stages, less energy is consumed. This is both more environmentally friendly, faster and cost efficient. In addition, as less drying is required, the taste and aroma is more retained.

The aerosol generating material that is produced by the process disclosed herein has a volatile content of between about 5% and about 30%. This enables the aerosol generating material to be cut into strips with relative ease. If the volatile content, in particular the water content, is too high, the aerosol-generating material may tear during the cutting process, which is undesirable. If the volatile content is too low, it may be too brittle and shatter during the cutting process.

In some embodiments, a further advantage is that a smaller amount of certain volatile components, in particular active agents and/or glycerol, are lost during drying of the mixture of the first and second compositions. Without wishing to be bound by reason, the mixture of the first composition and the second composition comprises a relatively low water content and as such requires less drying than a slurry that is known to the skilled person. For example, cooler temperatures and shorter drying times may be employed to achieve the desired volatile content of the aerosol-generating material. This also reduces the loss of certain valuable volatile components, resulting in improved flavor, taste and mouth-feel properties of the aerosol generated in the final product.

In some embodiments, the mixture of the first composition and the second composition comprises about 66% botanical material, about 17% aerosol former, about 8.2% filler and about 5% binder. In some embodiments, the botanical material is rooibos, the aerosol former is glycerol, the filler is cellulose, and the binder is CMC. It has been found according to the disclosure that this mixture of the first composition and the second composition provides the advantages disclosed herein.

The mixture of the first composition and the second composition, once formed and mixed, may be extruded using any extrusion technique or apparatus known in the art to from the aerosol generating material. Extrusion involves the feeding of a precursor composition through an orifice to produce an extruded agglomerate. The process, which applies pressure to the precursor composition combined with shear forces, results in agglomerated structures, which may be in the form of a sheet. Extrusion may be performed using one of the main classes of extruders: screw, sieve and basket, roll, ram and pin barrel extruders. Forming the sheet structures by extrusion has the advantage that this processing combines mixing, conditioning, homogenizing and moulding of the mixture of the first composition and the second composition.

Other materials may also be added during the extrusion process, such as a base, diluent, solid aerosol forming agents, solid flavor modifiers, expansion agents, active agent(s), and other additives known in the art. This has the advantage that the additive is evenly distributed throughout the agglomerated structures formed.

The resultant extruded mixture of the first composition and the second composition may be dried using any suitable drying technique known in the art. For example, microwave, infrared, air and oven drying are suitable techniques to dry the aerosol generating material. The temperature of the drying step may be below 100° C., and is below 90° C. in some embodiments. The drying temperature employed may be at most about 25° C., about 30° C., about 40° C., about 50° C., about 60° C., about 70° C., about 80° C., about 90° C., or about 100° C.

The resultant extruded mixture of the first composition and the second composition may be processed by forming a layer of the mixture on a surface and then the mixture may be dried to remove at least some of the water and form a sheet of the aerosol generating material. The water may be removed by allowing the water to evaporate from the extruded mixture at ambient temperature and pressure (for example, 25° C. and 101 kPa.) Alternatively, the water may be removed by applying heat to the extruded mixture (for example, by heating it to above about 25° C.) and/or reducing the atmospheric pressure surrounding the extruded mixture of the first composition and the second composition (for example, to less than 101 kPa).

A low drying temperature employed is advantageous as this reduces loss of volatile components, such as active agent, glycerol and flavors that contribute to the flavor, taste and mouth-feel of the final product. In some embodiments, there is a loss of less than about 10%, about 8%, about 5%, about 4%, about 2% or about 1% in total of active agent and glycerol. In some embodiments, there is less than 5% loss of total volatiles. In some embodiments, there is a loss of less than about 10%, about 8%, about 5%, about 4%, about 2% or about 1% in total of glycerol and active agent. In some embodiments, there is less than 5% loss in total of glycerol and active agent. The aerosol generating material therefore has a lower total volatile content than the mixture of the first composition and the second composition.

In some embodiments, the loss of water from the mixture of the first and second composition and the aerosol generating material is between 5-60%. In some embodiments, there is a loss of about 10%, about 15%, about 25%, about 30%, about 35%, about 40%, about 45%, about 50%, about 55% or about 60% of water. In some embodiments, there is a loss of about 40% to 55% water.

A sheet or shredded sheet of the aerosol generating material may comprise water and an aerosol former, in a total amount, of less than about 30% by weight of the sheet or shredded sheet of aerosol generating material or less than about 25% by weight of the aerosol generating material. It is thought that incorporating water and aerosol former in the sheet or shredded sheet of aerosol generating material in an amount of less than about 30% by weight of the sheet or shredded sheet of aerosol generating material may advantageously reduce the tackiness of the sheet. This may improve the ease by which the aerosol generating material can be handled during processing. For example, it may be easier to roll a sheet of aerosol-generating material to form a bobbin of material and then unroll the bobbin without the layers of sheet sticking together. Reducing the tackiness may also decrease the propensity for strands or strips of shredded material to clump or stick together, thus further improving processing efficiency and the quality of the final product.

The extruded mixture of the first composition and the second composition may be passed through one or a series of rollers to form the sheet of aerosol generating material having the desired thickness. In some embodiments, the distance between the rollers is different in different rolling presses. For example, the distance between the rollers may be progressively smaller to progressively flatten the material and control its thickness.

In some embodiments, the rollers are smooth. This provides the advantage of providing a sheet with reduced roughness and increased smoothness. In embodiments in which more than one rolling press is used, the use of repeated rolling over the material can further increase smoothness and decrease roughness.

In embodiments in which the rolling press(es) comprise two rollers, both the first and the second surface of the sheet may benefit from the increased smoothness and decreased roughness. This provides the additional advantage that the first and second side of the sheet or shredded sheet may be more consistent, and have a similar smoothness.

Reducing the thickness of the extruded mixture may shorten the drying period. The sheet may then be dried. After drying, the sheet of aerosol generating material may be cut into strips or strands of aerosol generating material. A single thickness of the sheet of aerosol generating material may be fed into a shredding apparatus. This can be achieved, for example, by providing a bobbin of sheet material which can be continuously fed into a shredding apparatus. Alternatively, a discrete portion of the aerosol generating material in sheet form, such as a sheet known to those skilled in the art as a flag, can be fed into a shredding apparatus. The strips or strands of aerosol generating material can be gathered and formed into an article for use in a non-combustible aerosol provision system. Optionally, the aerosol generating material can be crimped prior to being gathered and formed into the article. Optionally the aerosol generating material may be subject to a second cutting step, such as in a cross-cut type shredding process, to obtain a defined cut length.

The total volatile content may be about 5%, 10%, 15%, 20%, 25%, 30%, 40% by weight. The sheet or shredded sheet of aerosol generating material may have a total volatile content of about 5-15% by weight.

The composition of the aerosol contributes to the user's experience and satisfaction. One attribute that contributes to the user's experience and satisfaction is the presence of actives in the aerosol. Another attribute that contributes the user's experience and satisfaction is the perceived harshness of the aerosol. It is therefore important to control the actives content and harshness of an aerosol.

The first composition and/or the second composition may comprise the active agent (e.g., a substituted 3-(1-methylpyrrolidin-2-yl)pyridine as described herein). It may be preferable to add the active agent to the first composition. This enjoys the advantage that the active agent will be distributed evenly throughout at least the first composition.

In some embodiments, the first and second compositions are each completely free of substituted 3-(1-methylpyrrolidin-2-yl)pyridine. In some embodiments, the active agent (e.g., substituted 3-(1-methylpyrrolidin-2-yl)pyridine) may be added after forming the sheet, after drying the sheet, and/or after any shredding operation. For example, a nozzle may be provided on the machinery to deposit the active agent onto the surface of the sheet or strips of the aerosol generating material. In some embodiments the substituted 3-(1-methylpyrrolidin-2-yl)pyridine is applied as a solution in, for example, an aerosol former such as glycerol, propylene glycol, or a mixture thereof. Without wishing to be bound by theory, it is believed that adding the substituted 3-(1-methylpyrrolidin-2-yl)pyridine solely after sheet formation, either during or after drying or shredding, may reduce the potential for contamination and/or increase the homogeneity of distribution of the substituted 3-(1-methylpyrrolidin-2-yl)pyridine in the sheet or strips of aerosol generating material.

In some embodiments, the process involves adding an organic acid as described herein in either the first composition or the second composition. In some embodiments, the acid is selected from levulinic acid, lactic acid, benzoic acid, citric acid, 2-methylbutyric acid, and 2-methylvaleric acid. In some embodiments, the acid is benzoic acid. In some embodiments, the acid is levulinic acid.

When present, a flavorant may be added to any stage of the production of the aerosol generating material.

In some embodiments, additives may be incorporated into the second composition, the mixture of the first composition and the second composition or the aerosol generating material before or after extrusion, drying or shredding processing steps. In some embodiments, the additive comprises a substance to be delivered, such as an active agent or flavorant.

In some embodiments, the first composition, the second composition or the mixture of the first composition and the second composition may contain one or more functional materials as described herein above.

In one embodiment is provided a method of forming an aerosol generating material, the aerosol generating material comprising an aerosol former, a binder, a non-tobacco botanical material, and an active agent, the active agent comprising at least a substituted 3-(1-methylpyrrolidin-2-yl)pyridine, the method comprising mixing aerosol former, binder, non-tobacco botanical material, and active agent to form a slurry; and extruding the slurry through a die to form the aerosol generating material.

In another embodiment, an extrudate is form is noted above, and the extrudate is formed into beads, such as by processing the extrudate in a spheronizer.

In another embodiment, a fibrous material (e.g., a nonwoven material) is treated with substituted 3-(1-methylpyrrolidin-2-yl)pyridine, such as by spraying the active agent on the fibrous material or dipping the fibrous material in a liquid bath including the active agent. The fibrous material may also be treated with other components of an aerosol generating material as discussed herein, such as aerosol formers.

In another embodiment is provided a method of forming an aerosol generating material, the aerosol generating material comprising an aerosol former, a binder, a non-tobacco botanical material, and an active agent, the active agent comprising at least a substituted 3-(1-methylpyrrolidin-2-yl)pyridine, the method comprising mixing aerosol former, binder, and non-tobacco botanical material to form a slurry; extruding the slurry through a die to form an extrudate; and applying the active agent to the extrudate to form the aerosol generating material, such as by spraying the active agent onto the extrudate. As the extrusion process typically causes warming of the mixture during or prior to extrusion, application of the active agent after extrusion may be advantageous for active agents that are less thermally stable at the temperature of the extrusion process.

In another embodiment is provided a method of forming an aerosol generating material, the aerosol generating material comprising an aerosol former, a binder, a non-tobacco botanical material, and an active agent, the active agent comprising at least a substituted 3-(1-methylpyrrolidin-2-yl)pyridine, the method comprising mixing aerosol former, binder, and non-tobacco botanical material to form a slurry and casting the slurry onto a surface to form a sheet, which is optionally gathered or shredded thereafter. The substituted 3-(1-methylpyrrolidin-2-yl)pyridine can be sprayed on (or otherwise applied to) the sheet after formation, added to the slurry before sheet formation, or sprayed on (or otherwise applied to) a gathered or shredded form of the sheet material.

### Aerosol Generating Consumables and Devices

In another aspect is provided an aerosol generating consumable comprising an aerosol generating material as described herein. A consumable is an article comprising aerosol generating material, part or all of which is intended to be consumed during use by a user. A consumable may comprise one or more other components, such as an aerosol generating material storage area, an aerosol generating material transfer component, an aerosol generation area, a housing, a wrapper, a mouthpiece, and a filter. A consumable may also comprise an aerosol generator, such as a heater, that emits heat to cause the aerosol generating material to generate aerosol in use. The heater may, for example, comprise combustible material, a material heatable by electrical conduction, or a susceptor. The consumable may be any shape or size that is appropriate to the smoking device. In some embodiments of the disclosure, the consumable is a rod shape.

In another aspect is provided an aerosol generating device such as a tobacco-heating product (THP) or hybrid e-cigarette product for heating the aerosol generating material to volatilize at least one component of the aerosol generating material, also referred to herein as an “aerosol delivery system” or and “aerosol provision system.”. As used herein, the term “delivery system” is intended to encompass systems that deliver at least one substance to a user, and includes:
combustible aerosol provision systems, such as cigarettes, cigarillos, cigars, and tobacco for pipes or for roll-your-own or for make-your-own cigarettes (whether based on tobacco, tobacco derivatives, expanded tobacco, reconstituted tobacco, tobacco substitutes or other smokable material); and
non-combustible aerosol provision systems that release compounds from an aerosol-generating material without combusting the aerosol-generating material, such as electronic cigarettes, tobacco heating products, and hybrid systems to generate aerosol using a combination of aerosol-generating materials.

According to the present disclosure, a “combustible” aerosol provision system is one where a constituent aerosol generating material of the aerosol provision system (or component thereof) is combusted or burned during use in order to facilitate delivery of at least one substance to a user.

In some embodiments, the delivery system is a combustible aerosol provision system, such as a system selected from the group consisting of a cigarette, a cigarillo and a cigar.

In some embodiments, the disclosure relates to a component for use in a combustible aerosol provision system, such as a filter, a filter rod, a filter segment, or a paper such as a plug wrap, a tipping paper or a cigarette paper.

According to the present disclosure, a “non-combustible” aerosol provision system is one where a constituent aerosol generating material of the aerosol provision system (or component thereof) is not combusted or burned in order to facilitate delivery of at least one substance to a user.

In some embodiments, the delivery system is a non-combustible aerosol provision system, such as a powered non-combustible aerosol provision system.

In some embodiments, the non-combustible aerosol provision system is an electronic cigarette, also known as a vaping device or electronic nicotine delivery system (END), although it is noted that the presence of nicotine in the aerosol-generating material is not a requirement.

In some embodiments, the non-combustible aerosol provision system is an aerosol generating material heating system, also known as a heat-not-burn system. An example of such a system is a tobacco heating system.

In some embodiments, the non-combustible aerosol provision system is a hybrid system to generate aerosol using a combination of aerosol-generating materials, one or a plurality of which may be heated. Each of the aerosol-generating materials may be, for example, in the form of a solid, liquid or gel and may or may not contain nicotine. In some embodiments, the hybrid system comprises a liquid or gel aerosol generating material and a solid aerosol generating material, such as an aerosol generating material as described herein.

Typically, the non-combustible aerosol provision system may comprise a non-combustible aerosol provision device and a consumable for use with the non-combustible aerosol provision device.

In some embodiments, the disclosure relates to consumables comprising aerosol generating material and configured to be used with non-combustible aerosol provision devices. These consumables may sometimes be referred to as articles throughout the disclosure.

In some embodiments, the non-combustible aerosol provision system, such as a non-combustible aerosol provision device thereof, may comprise a power source and a controller. The power source may, for example, be an electric power source or an exothermic power source. In some embodiments, the exothermic power source comprises a carbon substrate which may be energized so as to distribute power in the form of heat to an aerosol generating material or to a heat transfer material in proximity to the exothermic power source.

In some embodiments, the non-combustible aerosol provision system may comprise an area for receiving the consumable, an aerosol generator, an aerosol generation area, a housing, a mouthpiece, and/or a filter.

In some embodiments, the consumable for use with the non-combustible aerosol provision device may comprise aerosol generating material, an aerosol generating material storage area, an aerosol generating material transfer component, an aerosol generator, an aerosol generation area, a housing, a wrapper, a filter, and/or a mouthpiece.

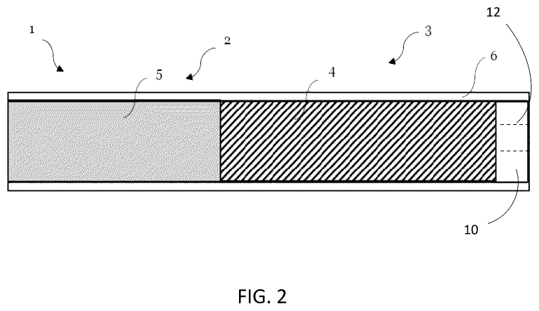

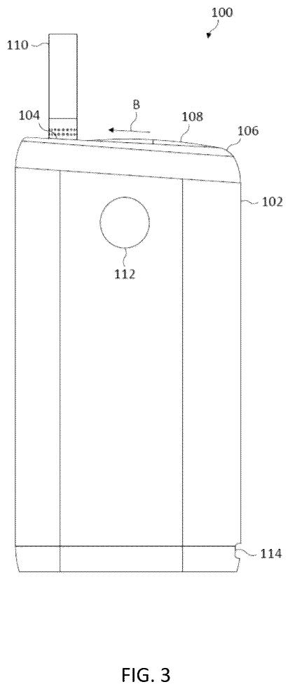

As disclosed herein, a non-combustible aerosol-provision system may comprise an aerosol generating material as disclosed herein. This is exemplified in FIG. 2 and FIG. 3.

FIG. 2 is a side-on cross-sectional view of a consumable or article 1 for use in an aerosol delivery system. The article 1 comprises a mouthpiece segment 2, and an aerosol-generating segment 3. The aerosol-generating segment 3 is in the form of a cylindrical rod and comprises an aerosol-generating material 4. The aerosol generating material can be any of the materials discussed herein. Although described above in rod form, the aerosol-generating segment 3 can be provided in other forms, for instance a plug, pouch, or packet of material within an article. The mouthpiece segment 2, in the illustrated embodiment, includes a body of material 5 such as a fibrous or filamentary tow. The rod-shaped consumable 1 further comprises a wrapper 6 circumscribing the mouthpiece segment 2 and aerosol generating segment 3, such as a paper wrapper.

The illustrated embodiment of FIG. 2 includes an optional end plug 10 at the upstream end of the consumable 1. The optional end plug 10 can serve to cover and protect all or a portion of the upstream surface of the aerosol generating material 4, which can, for example, minimize loss of aerosol former, active agent, and/or flavorant from the aerosol generating material prior to use. Example materials for the end plug 10 include sheet materials (e.g., sheet materials formed from one or more botanical materials), optionally in gathered form, foamed materials (e.g., cellulose foam), fibrous tow materials (e.g., cellulose acetate tow), and the like. In some embodiments, the end plug 10 comprises an optional central cavity 12 for receiving an aerosol generator (e.g., a pin heater) as described herein. The aerosol generating material 4 may also include a central cavity (not shown) that, for example, extends from the downstream end of the cavity 12 of the end plug 10.

In some embodiments, the end plug 10 does not comprise a central cavity, but is configured to deform to allow receipt of an aerosol generator (e.g., a pin heater). For example, in some embodiments, the end plug is in a sheet form and is configured such that the sheet material is easily deformed to allow penetration of the aerosol generator.

FIG. 3 shows an example of a non-combustible aerosol provision device 100 for generating aerosol from an aerosol generating medium/material such as the aerosol generating material of a consumable 110, as described herein. In broad outline, the device 100 may be used to heat a replaceable article 110 comprising the aerosol-generating medium, for instance an article 1 as illustrated in FIG. 2 or as described elsewhere herein, to generate an aerosol or other inhalable medium which is inhaled by a user of the device 100. The device 100 and replaceable article 110 together form a system.

The device 100 comprises a housing 102 (in the form of an outer cover) which surrounds and houses various components of the device 100. The device 100 has an opening 104 in one end, through which the article 110 may be inserted for heating by a heating assembly. In use, the article 110 may be fully or partially inserted into the heating assembly where it may be heated by one or more components of the heater assembly.

The device 100 of this example comprises a first end member 106 which comprises a lid 108 which is moveable relative to the first end member 106 to close the opening 104 when no article 110 is in place. In FIG. 3, the lid 108 is shown in an open configuration, however the lid 108 may move into a closed configuration. For example, a user may cause the lid 108 to slide in the direction of arrow “B”.

The device 100 may also include a user-operable control element 112, such as a button or switch, which operates the device 100 when pressed. For example, a user may turn on the device 100 by operating the switch 112.

The device 100 may also comprise an electrical component, such as a socket/port 114, which can receive a cable to charge a battery of the device 100. For example, the socket 114 may be a charging port, such as a USB charging port.

In some embodiments, the device may also comprise an aerosol generator. An aerosol generator is an apparatus configured to cause aerosol to be generated from the aerosol generating material. In some embodiments, the aerosol generator is a heater configured to subject the aerosol-generating material to heat energy, so as to release one or more volatiles from the aerosol generating material to form an aerosol. In some embodiments, the aerosol generator is configured to cause an aerosol to be generated from the aerosol-generating material without heating. For example, the aerosol generator may be configured to subject the aerosol-generating material to one or more of vibration, increased pressure, or electrostatic energy.

Example aerosol generator technologies include an induction heater, a laser heater, a plasma heater, a microfluidic heater, a convection heater, allotrope of carbon heater, carbon foam heater, a radio-frequency heater, an electro-resistive heater, and a halogen heater. An example aerosol generator is commercially available as the GLO™ Hyper Pro device.

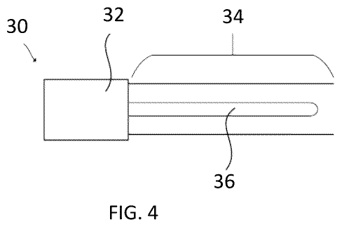

As shown FIG. 4, the components of an embodiment of a non-combustible aerosol provision device 30 are shown in a simplified manner. Particularly, the elements of the non-combustible aerosol provision device 30 are not drawn to scale in FIG. 4. Elements that are not relevant for the understanding of this embodiment have been omitted to simplify FIG. 4.

The non-combustible aerosol provision device 30 comprises a housing 32 comprising an area 34 for receiving an article. The area is in the form of a cavity, open at the proximal end (or mouth end) for receiving an aerosol-generating article, such as the consumable 1 of FIG. 2. The distal end of the cavity is spanned by an aerosol-generating assembly comprising an aerosol generator 36. In this embodiment, the aerosol-generator is in the form of a resistively-heated elongate pin.

The aerosol generator 36 is retained by a heater mount (not shown) such that an active heating area of the aerosol generator is located within the cavity. The active heating area of the aerosol generator 36 is positioned, for example, within one or both of the end plug 10 and the aerosol-generating material 4 of consumable 1 of FIG. 2 when the consumable is fully received within the cavity.

The aerosol generator 36 is configured for insertion through a pierceable embodiment of end plug 10 of FIG. 2 or into a central cavity in the end plug, and may also extend into an aerosol-generating material 4 of consumable 1 of FIG. 2. As noted above, the aerosol generator 36 is shaped in the form of a pin terminating in a rounded point. The pin has a length dimension that is greater than its width dimension.

Many modifications and other implementations of the disclosure will come to mind to one skilled in the art to which this disclosure pertains having the benefit of the teachings presented in the foregoing descriptions and the associated figures. Therefore, it is to be understood that the disclosure is not to be limited to the specific implementations disclosed herein and that modifications and other implementations are intended to be included within the scope of the appended claims. Although specific terms are employed herein, they are used in a generic and descriptive sense only and not for purposes of limitation.

### EXAMPLE

### Example 1. Aerosol Generating Material

An aerosol generating material according to a noon-limiting embodiment of the disclosure is be prepared by forming a first composition (“liquid phase”) comprising binder, an active agent (optional), an acid (optional), an aerosol former, and water; and forming a second composition (“dry phase”) comprising a non-tobacco botanical material and a filler; and combining the first composition and the second composition. The mixture is then extruded, rolled between a pair of rollers to form a sheet and the sheet is dried at less than 100° C. to form dried sheets of aerosol generating material. The composition of the aerosol generating material is provided in Table 2.

TABLE 2
Composition of aerosol generating material

| | | Mixture A | Mixture B |
|---|---|---|---|
| Component | | (dwb*) | (dwb) |
| Botanical material | (e.g., Rooibos) | 68.6 | 66% |
| Aerosol former | (e.g., Glycerin) | 17.7 | 17% |
| Filler | (e.g., Cellulose) | 8.5 | 8.2% |
| Binder | (e.g., CMC) | 5.2 | 5% |
| Active | substituted 3-(1- | 0% | 2% |
| | methylpyrrolidin-2- | | |
| | yl)pyridine | | |
| Acid | (e.g., Benzoic acid) | 0% | 1.8% |
| Total | | 100% | 100% |

“dwb” = dry weight basis

1. An aerosol generating material for use in a non-combustible aerosol provision system, wherein the aerosol generating material is in sheet form, shredded form, bead form, fibrous form, or extrudate form, the aerosol generating material comprising an aerosol former and an active agent, the active agent comprising at least a compound having a structure according to Formula I, Formula II, or Formula III: 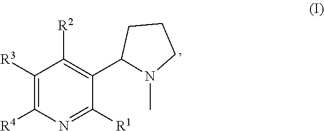
wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and wherein at least one of R¹, R², R³, and R⁴ are not hydrogen; or 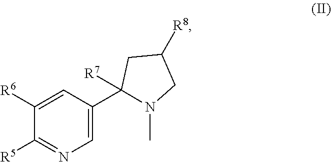
wherein:
R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
R⁷ is selected from the group consisting of hydrogen and CH₃;
R⁸ is selected from the group consisting of hydrogen and C1-C3 alkyl; and
at least one of R⁷ and R⁸ is not hydrogen; or 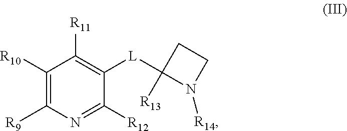
wherein
L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.

2. The aerosol generating material of claim 1, wherein the active agent comprises a compound having a structure according to Formula I, wherein R¹, R², and R³ are each H.

3. The aerosol generating material of claim 2, wherein R⁴ is optionally substituted C₁-C₆ alkyl, F, Cl, Br, OCH₃, OEt, or CN.

4. The aerosol generating material of claim 3, wherein R⁴ is C₁-C₃ alkyl, such as wherein R⁴ is CH₃.

5. The aerosol generating material of claim 1, wherein the active agent comprises a compound having a structure according to Formula II, wherein R⁵ and R⁶ are H; R⁷ is CH₃; and R⁸ is H.

6. The aerosol generating material of claim 1, wherein the active agent comprises a compound having a structure according to Formula II, wherein R⁵, R⁶ and R⁷ are H, and R⁸ is CH₃.

7. The aerosol generating material of claim 1, wherein the aerosol generating material further comprises a binder and a non-tobacco botanical material, optionally wherein the non-tobacco botanical material comprises fennel, star anise, hemp, rooibos, or a combination thereof.

8. The aerosol generating material of claim 1, wherein the aerosol generating material further comprises a filler.

9. The aerosol generating material of claim 1, wherein the aerosol generating material is in the form of a sheet or a shredded sheet.

10. The aerosol generating material of claim 1, wherein the aerosol generating material is in the form of a plurality of beads.

11. The aerosol generating material of claim 1, wherein the aerosol generating material is in an extruded solid form, optionally wherein the extruded solid form comprises one or more passages therethrough to allow air to pass through the extruded solid form, and optionally wherein the extruded solid form has a cavity adapted to receive an aerosol generator, such as a heater.

12. The aerosol generating material of claim 11, further comprising an end plug adjacent to the extruded solid aerosol generating material, optionally wherein the end plug has a cavity adapted to receive an aerosol generator, such as a heater.

13. The aerosol generating material of claim 1, wherein the aerosol generating material is in a fibrous form, such as in the form of a nonwoven material, optionally wherein the nonwoven material comprises regenerated cellulose.

14. The aerosol generating material of claim 1, wherein the active agent further comprises a nicotine component, a cannabinoid, a terpene, caffeine, an amino acid, a vitamin, melatonin, a botanical extract, or a combination thereof.

15. The aerosol generating material of claim 1, wherein the active agent further comprises a nicotine component.

16. The aerosol generating material of claim 1, wherein the aerosol generating material is substantially free of a nicotine component.

17. The aerosol generating material of claim 1, wherein the aerosol generating material further comprises a flavorant.

18. The aerosol generating material of claim 1, wherein the aerosol generating material is substantially free of tobacco material.

19. An aerosol provision system comprising the aerosol generating material of claim 1.

20. A method of forming an aerosol generating material in sheet form, shredded form, bead form, or extrudate form, the aerosol generating material comprising an aerosol former, a binder, a non-tobacco botanical material, and an active agent, the active agent comprising at least a substituted 3-(1-methylpyrrolidin-2-yl)pyridine, 3-(azetidin-2-yl)pyridine, or 3-(azetidin-2-ylmethoxy)pyridine, the method comprising:
mixing aerosol former, binder, non-tobacco botanical material, and active agent to form a slurry; and
extruding the slurry through a die to form the aerosol generating material.

21. A method of forming an aerosol generating material in sheet form, shredded form, bead form, or extrudate form, the aerosol generating material comprising an aerosol former, a binder, a non-tobacco botanical material, and an active agent, the active agent comprising at least a substituted 3-(1-methylpyrrolidin-2-yl)pyridine, 3-(azetidin-2-yl)pyridine, or 3-(azetidin-2-ylmethoxy)pyridine, the method comprising:
mixing aerosol former, binder, and non-tobacco botanical material to form a slurry;
extruding the slurry through a die to form an extrudate; and
applying the active agent to the extrudate to form the aerosol generating material.
